(19) United States
(12) Patent Application Publication
(10) Pub. No.: US 2025/0277217 A1
(43) Pub. Date: Sep. 4, 2025

(54) METHODS AND COMPOSITIONS FOR TREATING INFLAMMATORY CONDITIONS ASSOCIATED WITH INFECTIOUS DISEASE

(71) Applicant: Direct Biologics, LLC, Austin, TX (US)

(72) Inventors: Kenneth Allen Pettine, Fort Collins, CO (US); Kevin Hicok, San Diego, CA (US); Timothy Alexander Moseley, Fallbrook, CA (US)

(21) Appl. No.: 19/098,197

(22) Filed: Apr. 2, 2025

Related U.S. Application Data
(63) Continuation of application No. 17/920,997, filed on Oct. 24, 2022.
(60) Provisional application No. 63/013,865, filed on Apr. 22, 2020.
(60) Provisional application No. 63/198,706, filed on Nov. 6, 2020.

(51) Int. Cl. C12N 15/113; A61K 9/127; A61K 38/17; A61K 45/06; A61P 31/14

(52) U.S. Cl. CPC C12N 15/1137; A61K 9/127; A61K 38/1709; A61K 38/177; A61K 45/06; A61P 31/14; C12N 15/1136; C12N 2310/141

(57) ABSTRACT

Disclosed herein are compositions comprising a mesenchymal stem cell (MSC) preparation and one or more biomolecules and the use of said compositions for the treatment or a microbial infection or the symptoms or secondary pathological conditions associated with said infection.

This application is a continuation of U.S. application Ser. No. 17/920,997, filed on Oct. 24, 2022, which is a 371 U.S. National Stage of International Application No. PCT/US2021/028686, filed on Apr. 22, 2021, which claims the benefit of U.S. Provisional Application No. 63/198,706, filed on Nov. 6, 2020, and U.S. Provisional Application No. 63/013,865, filed on Apr. 22, 2020, each of which is incorporated herein by reference in its entirety.

### I. BACKGROUND

There are currently no effective biologic treatments for coronaviruses, especially for COVID-19. Commercially available products do not act concurrently and synergistically to directly affect viral infection and/or replication as well as to regulate downstream inflammation and vascular cell related pathologies in response to the viral infection. Therefore, what is needed is a method and composition for an intravenous or pulmonary route of delivery of autogenous or allogenic MSC derived growth factors and exosomes, wherein the products are acellular and useful for the treatment of various viruses, while simultaneously avoiding the potential negative long-term effects associated with introducing cellular living MSCs into a recipient.

### II. SUMMARY

Disclosed are methods and compositions related to a composition comprising MSC secretome (such as, for example, including, but not limited to MSC growth factor, MSC exosome, MSC extracts and/or extracellular vesicle comprising compositions) and/or one or more biomolecules for use treating diseases.

In one aspect, disclosed herein are compositions comprising a therapeutically effective amount of a MSC secretome (such as, for example, including, but not limited to MSC growth factor, MSC exosome, MSC extracts and/or extracellular vesicle comprising compositions) and one or more biomolecules (such as, for example, a peptide, polypeptide, protein, siRNA, shRNA, and/or microRNA (miRNA)) that selectively bind to one or more microbial immunogens, or inhibit the ability of a microbe to inhabit a host, or inhibit, decrease, reduce, ameliorate, and/or prevent one or more secondary conditions caused by a microbial infection (such as, for example, comprise a peptide, polypeptide, protein, siRNA, shRNA, microRNA (miRNA) which act concurrently and synergistically to directly affect viral infection and/or replication and/or act concurrently and synergistically to regulate downstream inflammation and vascular cell related pathologies in response to microbial infection). For example, disclosed herein are compositions comprising a therapeutically effective amount of a MSC secretome (such as, for example, including, but not limited to MSC growth factor, MSC exosome, MSC extracts and/or extracellular vesicle comprising compositions) and one or more biomolecules comprise a ferritin protein, PAI-1, thrombomodulin, and/or a miRNA is selected from the group of miRNA comprising hsa-let-7a-5p, hsa-let-7b-5p, hsa-let-7c-5p, hsa-let-7d-3p, hsa-let-7e-5p, hsa-let-7g-5p, hsa-let-7i, hsa-let-7i-5p, hsa-miR-100-5p, hsa-miR-103a-3p, hsa-miR-106a-5p, hsa-miR-106b-5p, hsa-mir-10b, hsa-miR-10b-5p, hsa-mir-1246, hsa-miR-1246, hsa-miR-125a-5p, hsa-miR-125b-5p, hsa-miR-130a-3p, hsa-mir-130b, hsa-miR-130b-3p, hsa-miR-132-3p, hsa-miR-136-5p, hsa-miR-138-5p, hsa-miR-139-5p, hsa-mir-140, hsa-miR-140-3p, hsa-miR-145-5p, hsa-mir-146a, hsa-miR-146a-5p, hsa-miR-148a-3p, hsa-miR-152-3p, hsa-miR-15a-5p, hsa-miR-15b-5p, hsa-mir-16-1, hsa-mir-16-2, hsa-miR-16-5p, hsa-miR-17-5p, hsa-miR-181a-5p, hsa-miR-191-5p, hsa-miR-193a-5p, hsa-miR-193b-3p, hsa-miR-197-3p, hsa-miR-199a-3p, hsa-miR-199a-5p, hsa-miR-199b-5p, hsa-miR-19a-3p, hsa-miR-19b-3p, hsa-miR-20a-5p, hsa-mir-203a, hsa-miR-203a-3p, hsa-miR-214-3p, hsa-mir-21, hsa-miR-21-3p, hsa-miR-21-5p, hsa-mir-221, hsa-miR-221-3p, hsa-mir-222, hsa-miR-222-3p, hsa-miR-22-3p, hsa-miR-23a-3p, hsa-miR-23b-3p, hsa-mir-24-1, hsa-mir-24-2, hsa-miR-24-3p, hsa-mir-25, hsa-miR-25-3p, hsa-miR-26a-5p, hsa-miR-27a-3p, hsa-mir-27b, hsa-miR-27b-3p, hsa-miR-29a-3p, hsa-miR-29c-3p, hsa-miR-30a-5p, hsa-miR-30a-5p, hsa-miR-30b-5p, hsa-miR-30c-5p, hsa-mir-30d, hsa-miR-30d-5p, hsa-mir-30e, hsa-miR-30e-5p, hsa-miR-31-3p, hsa-miR-31-5p, hsa-miR-320a, hsa-miR-342-3p, hsa-miR-345-5p, hsa-miR-34a-5p, hsa-miR-361-5p, hsa-miR-376a-3p, hsa-miR-376c-3p, hsa-miR-423-3p, hsa-miR-423-5p, hsa-miR-424-5p, hsa-miR-484, hsa-mir-486-1, hsa-mir-486-2, hsa-miR-486-5p, hsa-miR-570-3p, hsa-miR-574-3p, hsa-miR-663a, hsa-miR-874-3p, hsa-mir-92a-1, hsa-mir-92a-2, hsa-miR-92a-3p, hsa-miR-92b-3p, hsa-mir-93, hsa-miR-93-5p, hsa-miR-940, hsa-miR-99a-5p, and hsa-miR-99b-5p.

Also disclosed herein are compositions of any preceding aspect, wherein the one or more biomolecules inhibit the bradykinin pathway (for example, by inhibiting translation of Bradykinin 2), angiotensin-converting enzyme 2 (ACE 2) receptor, inhibits the transmembrane protease, seine 2 (TMPRSS2) enzyme, inhibits Kallikrein B1, and/or inhibits IL-1β, IL-6, TNF-α, GM-CSF, or M-CSF.

In one aspect, disclosed herein are compositions of any preceding aspect, wherein the MSC secretome further comprises prostaglandin E2 (PGE2), transforming growth factor β31 (TGF-β1), hepatocyte growth factor (HGF), stromal cell derived factor-1 (SDF-1), nitric oxide, indoleamine 2,3-dioxygenase, interleukin-4 (IL-4), IL-6, interleukin-10 (IL-10), IL-1 receptor antagonist and soluble TNF-α receptor, insulin-like growth factors, fibroblast growth factors (FGF) 1-23 (especially, FGF1 and FGF2), bone morphogenetic proteins (BMPs) 1-15, epidermal growth factor (EGF), transforming growth factor-α (TGF-α) macrophage-stimulating protein (MSP), platelet derived growth factor (PLGF), vascular endothelial growth factor (VEGF), macrophage colony stimulating factor (M-CSF), insulin, granulocyte colony stimulating factor (G-CSF), granulocyte macrophage colony stimulating factor (GM-CSF estrogen, and/or thyroid hormones.

Also disclosed herein are methods of treating, decreasing, inhibiting, reducing, ameliorating and/or preventing a microbial infection (such as, for example, a viral, bacterial, fungal, or parasitic infection) or symptoms thereof (including, but not limited to microbial induced cytokine storm, microbial initiated bradykinin storm, and/or acute respiratory distress syndrome) in a subject comprising administering to a subject the composition of any preceding aspect.

In one aspect, disclosed herein are methods of treating, decreasing, inhibiting, reducing, ameliorating and/or preventing a microbial infection or symptoms thereof of any preceding aspect, wherein the microbial infection comprises an infection from a virus selected from the group of viruses consisting of Herpes Simplex virus- 1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2, or MERS-CoV), Influenza virus A, Influenza virus B, Measles virus, Polyomavirus, Human Papillomavirus, Respiratory syncytial virus, Adenovirus, Coxsackie virus, Chikungunya virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

Also disclosed herein are methods of treating, decreasing, inhibiting, reducing, ameliorating and/or preventing a microbial infection or symptoms thereof of any preceding aspect, wherein the microbial infection comprises an infection from a bacteria selected from the group of bacteria consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Mycobacterium chimaera, Nocardia asteroides, other Nocardia species, Legionella pneumophila, other Legionella species, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella bumetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species, and Mycoplasma species. In one aspect the bacteria is not Bacillus anthracis.

In one aspect, also disclosed herein are methods of treating, decreasing, inhibiting, reducing, ameliorating and/or preventing a microbial infection or symptoms thereof of any preceding aspect, wherein the microbial infection comprises an infection from a fungus selected from the group of fungi consisting of Candida albicans, Cryptococcus neoformans, Histoplasma capsulatum, Aspergillus fumigatus, Coccidiodes immitis, Paracoccidioides brasiliensis, Blastomyces dermitidis, Pneumocystis carinii, Penicillium marneffi, and Alternaria altemata.

Also disclosed herein are methods of treating, decreasing, inhibiting, reducing, ameliorating and/or preventing a microbial infection or symptoms thereof of any preceding aspect, wherein the microbial infection comprises a parasitic infection with a parasite selected from the group of parasitic organisms consisting of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, and Entamoeba histolytica.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A, 1B, and 1C show a set of representative images captured by transmission electron microscopy (TEM) showing size distribution of EVs present within a sample of a composition according to an embodiment of the invention;

FIGS. 2A, 2B, 2C, and 2D are a set of charts representing numbers of captured exosome particles;

FIGS. 3A, 3B, 3C, and 3D are a set of microphotographic images of CD63 captured exosomes;

FIG. 4 is a table from Park et al. (2020) BioRxiv showing miRNAs predicted to bind the SARS-CoV-2 RNA genome FIG. 5 is a schematic representation of multiple mechanisms of action of the inventive composition; and

FIG. 6 is a graph demonstrating a direct effect of the composition of FIG. 1 on live CoV-2 virus replication.

FIG. 7 is a compilation of graphs demonstrating the effect of the invention when administered to patients with severe COVID-19 related ARDS. Response of COVID-19 patients to intravenous administration of invention. “Acute phase reactants” (CRP, ferritin, and D-dimer) and immune cell populations on day of treatment before IV administration and on day 5 post-treatment. Mean reductions of CRP, ferritin, and D-dimer reductions were 77% (P<0.001), 43% (P<0.001), and 42% (P<0.05), respectively. Mean reduction of ANC was 32% (P<0.001); Total lymphocyte count increased by 36% (P<0.05) with CD3+, CD4+, and CD8+T lymphocytes increased by 46% (P<0.05), 45% (P<0.05), and 46% (P<0.001), respectively. ANC, absolute neutrophil count; CRP, C-reactive protein.

FIG. 8 is a graph illustrating one mechanism of action is to inhibit the master inflammation inducing cytokine, IL-10.

FIG. 9 is a graph illustrating one mechanism of action is to regulate a hyper-active immune response by neutrophils by decreasing neutrophil extracellular trap formation (Netosis).

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

The term “subject” is defined herein to include animals such as mammals, including, but not limited to, primates (e.g., humans), cows, horses, pigs, sheep, goats, dogs, cats, rabbits, rats, mice and the like. In some embodiments, the subject is a human.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intraarticular, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily, or the dose may be proportionally reduced as indicated by the exigencies of the therapeutic situation.

A “decrease” can refer to any change that results in a smaller gene expression, protein production, amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease, disorder, injury, or condition, or an underlying cause of a disease or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of an infection.

The terms “prevent,” “preventing,” “prevention,” and grammatical variations thereof as used herein, refer to a method of partially or completely delaying or precluding the onset or recurrence of a disease and/or one or more of its attendant symptoms or barring a subject from acquiring or reacquiring a disease or reducing a subject's risk of acquiring or reacquiring a disease or one or more of its attendant symptoms.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity.

Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain (i.e., nociception) relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular MSC secretome (including, but not limited to a MSC exosome (with or without growth factors) referred to herein as an extracellular vesicle isolate product (EVIP)) is disclosed and discussed and a number of modifications that can be made to a number of molecules including the MSC secretome are discussed, specifically contemplated is each and every combination and permutation of MSC secretome and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Currently, COVID-19 is ravaging the world. It has been widely reported that children are less likely to get severely ill and die from COVID-19. A recent study of 44,672 people with confirmed COVID-19 infection found that children under 10 years old made up less than 1 percent of those cases and none of the 1,023 deaths. A straightforward explanation would be that children are resisting infection in the first place, but that does not seem to be the case. Children are just as likely as adults to get infected. So, what is protecting children? Experts suspect it may be because of the unique way children's immune systems respond to these viruses.

A common complication of COVID-19, SARS and MERS in adults is where the immune response against the coronavirus becomes overzealous and causes life-threatening damage to the lungs. The resulting leakage of fluid and immune cells into the lungs can result in life threatening acute respiratory distress syndrome (ARDS) problems. Even if those immune responses are trying to help by attacking the virus, they can end up blocking oxygen uptake in the lungs. Because children's immune systems are still developing, one suggestion is that they are shielded from this type of dangerous immune response, called a cytokine storm, when they get COVID-19 or similar diseases.

Airway epithelial cell injury is also important in COVID-19 pathogenesis. Injured lung epithelial cells are a source of inflammatory mediators such as IL-6, TNF-α, IL-1β, GM-CSF, and CXCL-8, which may act in both autocrine and paracrine manners. IL-6 induces the remodeling of airway cells by regulation and promoting of myofibroblast differentiation which is the main cause of fibrosis development during airway remodeling. The paracrine activity of these mediators is mediated, at least in part, by inflammatory exosomes.

Acute inflammation is a key pathological feature of the COVID-19 process. For example, cytokines produced by natural killer cells, mast cells, macrophages, and monocytes at the site of inflammation play a key role in the development of the COVID-19 cytokine storm that results in ARDS. Proinflammatory biomarkers, such as cytokines, have been found in both chronic and acute pulmonary disease states, suggesting either a direct or faciliatory role in the occurrence of the pathology. Multiple cytokines are produced during an inflammatory reaction. Cytokines contribute to inflammatory processes by activation of specific signal transduction mechanisms as well as the activation of other cell types. Cytokines are found extracellularly (in blood) and in interstitial compartments, where they can activate cells in an autocrine/paracrine fashion. It has been postulated that increased levels of cytokines influence and contribute to COVID-19 respiratory symptoms by increasing the sensitization of nociceptors. When tissue is invaded or destroyed by pulmonary leukocytes during an inflammatory episode, several mediators such as interleukin-1 (IL-1), IL-8, IL-6, and tumor necrosis factor alpha (TNF-α) migrate to the site. Also included in these mediators are nerve growth factor and prostaglandins. These all result in pulmonary inflammation.

Mesenchymal stem cells (MSCs) have attracted much attention for their ability to regulate inflammatory processes. In many types of pulmonary trauma, inflammatory conditions at the site of injury impede the natural repair processes by local progenitor and mature cells. MSCs assist via paracrine mechanisms and modulate the regenerative environment via anti-inflammatory and immunomodulatory mechanisms. In response to inflammatory molecules such as interleukin-1 (IL-1), IL-2, 11-6, IL-12, tumor necrosis factor-a (TNF-α) and interferon-gamma (INF-γ), MSCs secrete an array of growth factors and anti-inflammatory proteins with complex feedback mechanisms among the many types of immune cells. The key immunomodulatory cytokines include prostaglandin 2, TGF-β1, HGF, SDF-1, indoleamine 2,3-dioxygenase, IL-4, IL-1β, IL-1 receptor antagonist and soluble tumor necrosis factor-a receptor. MSCs prevent proliferation and the dysfunction of many inflammatory immune cells, including T cells, natural killer cells, B cells, monocytes, macrophage, and dendritic cells.

The primary trophic property of MSCs is the secretion of growth factors and exosomes to induce cell proliferation and angiogenesis. Exosomes express mitogenic proteins such as transforming growth factor-alpha (TGF-α), TGF-β, hepatocyte growth factor (HGF), epithelial growth factor (EGF), basic fibroblast growth factor (FGF-2) and insulin-like growth factor-1 (IGF-1). These increase fibroblast, epithelial and endothelial cell division. Vascular endothelial growth factor (VEGF), IGF-1, EGF and angiopoietin-1 are released to recruit endothelial lineage cells and initiate vascularization.

MSCs assist via paracrine mechanisms and modulate the regenerative environment via anti-inflammatory and immunomodulatory mechanisms. In response to inflammatory molecules such as interleukin-1 (IL-1), IL-6, IL-2, IL-12, tumor necrosis factor-α (TNF-α) and interferon-gamma (INF-γ), MSCs secrete an array of growth factors and anti-inflammatory proteins with complex feedback mechanisms among the many types of immune cells. The key immunomodulatory cytokines include prostaglandin 2, TGF-β1, HGF, SDF-1, nitrous oxide, indoleamine 2, 3-dioxygenase, IL-4, IL-1β, IL-1 receptor antagonist and soluble tumor necrosis factor-α receptor. MSCs prevent proliferation and function of many inflammatory immune cells, including T-cells, natural killer cells, B-cells, monocytes, macrophages, and dendritic cells. Although MSCs across species are able to regulate T-cell activity, the mechanisms are not identical across mammalian species.

A characteristic of chronically inflamed environments is a persistent imbalance in the types of helper T-cells and macrophages. MSC exosomes indirectly promote the transition of TH1 to TH2 cells by reducing INF-γ and increasing IL-4 and IL-10. The restored TH1/TH2 balance has been shown to improve tissue regeneration in cartilage, muscle, and other soft tissue injuries, alleviate symptoms of autoimmune diseases, and have an anti-diabetic effect. Similarly, reduction in INF-γ and secretion of IL-4 promotes a shift in macrophages from M1 (proinflammatory, anti-angiogenic and tissue growth inhibition) to M2 (anti-inflammatory, pro-remodeling and tissue healing) type, an effect required for skeletal, muscular, and neural healing and regeneration.

Disclosed herein is a complex composition of secreted biomolecules (proteins, lipids, and ribonucleic acids) and/or extracellular vesicles comprising biomolecules, originating from mesenchymal lineage cells, that interact directly with microbial proteins (such as, for example, a coronavirus protein) and/or nucleic acid content (such as, for example microbial ribonucleic acid content) to disrupt the capacity of the microbe to establish an infection in the subject, infect cells, and/or replicate within cells, and that concurrently interact with infected cells and immune response cells to resolve hyperactive responses (such as for example, microbial induced cytokine storm, microbial initiated bradykinin storm, and/or acute respiratory distress syndrome). In one aspect, disclosed herein are compositions comprising a therapeutically effective amount of a MSC secretome (such as, for example, including, but not limited to MSC growth factor, MSC exosome, MSC extracts and/or extracellular vesicle comprising compositions) and one or more biomolecules (such as, for example, a peptide, polypeptide, protein, siRNA, shRNA, and/or microRNA (miRNA)) that selectively bind to one or more microbial immunogens, or inhibit the ability of a microbe to inhabit a host, or inhibit, decrease, reduce, ameliorate, and/or prevent one or more secondary conditions caused by a microbial infection (such as, for example, comprise a peptide, polypeptide, protein, siRNA, shRNA, microRNA (miRNA) which act concurrently and synergistically to directly affect viral infection and/or replication and/or act concurrently and synergistically to regulate downstream inflammation and vascular cell related pathologies in response to microbial infection).

In some embodiments, the composition comprises bone marrow-derived mesenchymal lineage cells that adhere to culture plastic and may differentiate in culture into multiple mature cell fates including but not limited to adipocyte, osteoblast and chondrocyte fates and/or secreted extracellular vesicles that contain a core protein composition that includes any combination of core composition proteins, selected from the group consisting of. Ferritin, NUP85, LAMP2, GPR115, Serpin F1, OPN, PAI-1, DAPP1, Cathepsin B, Semaphorin 6C, PDGF R alpha, Sortilin, Serpin B6, Dkk-3, Thrombomodulin, PF4, MIF, Periostin, Furin, TIMP-1, Decorin, PCK1, CD99, CD63, CD9, CD81, Transferrin, DcR3, Lumican, TIMP-2, SLITRK5, FAP, Artemin, DPPII, cIAP-1, Pentraxin 3, Visfatin, Neprilysin, Albumin, Galectin-1, UNC5H3, IL-20 R beta, SREC-II, JAM-C, TNF RI, htPAPP-A, eNOS, MSP R, TPP1, LAMP1, B2M, NCAM-1, HIF-1 alpha, ST6GAL1, CD99-L2, Plexin A4, EMMPRIN, p53, Semaphorin 7A, NKp80, Cystatin B, Osteoadherin, Midkine, Calreticulin, Osteoactivin, Legumain, TAZ, Cathepsin L, RBP4, Serpin A4, JAM-A, MCSF, LIMPII, OPG, IL-22, Galectin-3, MOG, Trypsin 3, SIRP alpha, and Syndecan-4, and at least one protein selected from the group consisting of: Ferritin, IGFBP-4 IL-1 R6 GSTM1, NUP85, LAMP2, MeprinA, IL-1 F10, bIG-H3, GPR115, TGFb1, Ephrin-A4, CD109, Serpin F1, IGFBP-6, HS3ST4, Aminopeptidase LRAP, OPN, PAI-1, DAPP1, GDF-9, Cathepsin B, IGFBP-2, Semaphorin 6C, IGF-2, PDGF R alpha, Sortilin, Serpin B6, Dkk-3, CNTF, TSP-1, GM-CSF Ra, Thrombomodulin, Endoglycan, IGFBP-3, RGM-C, PF4, MIF, TGM4, Periostin, Furin, TIMP-1, PAPP-A, Decorin, PCK1, Arylsulfatase A, CD99, CA2, PRDX4, Transferrin, DcR3, GP73, LAIR2, ULBP-4, Lumican, TIMP-2, TFPI, SOX2, SLITRK5, FAP, Spinesin, ENPP-2, CD97, CTACK, Integrin alpha 1, EXTL3, IL-18 BPa, PD-L2, PSMA, IL-20 Ra, Glyoxalase II, Trypsin 1, IGF-2R, ADAMTSL-1, Erythropoietin, Plexin D1, DNMT3A, BCL-2, CL-P1, Ephrin-B3, FABP6, CHI3L1, FCRL5, TFF3, Artemin, DPPII, cIAP-1, PDGF Rb, Pentraxin 3, Angiotensinogen, Follistatin, CF VII, Persephin, TRAIL RI, THAP11, CD200, CLEC-2, AMIGO, IGFBP-5, PON1, SOX7, GALNT10, Visfatin, Progranulin, PCSK2, GKN1, IL-18, Neprilysin, Stabilin-2, IL-17 RD, Albumin, Follistatin-like 1, MMP-10, FKBP51, LRRC4, Pref-1, Galectin-1, Troponin C, UNC5H3, FLRT2, CD314, Semaphorin 6B, Netrin-4, CD27 Ligand, IL-20 R beta, Semaphorin 6A, TSK, Cytokeratin-8, CHST3, Mcl-1, DPPIV, SREC-II, Norrin, JAM-C, Bcl-10, Wnt-4, LSECtin, Kell, TNF RI, PTP1B, htPAPP-A, IDO, PDGF-CC, Galanin, Activin A, TLR2, SCCA2, FABP1, eNOS, SHP-1, ICOS, ClqTNF9, MMP-1, TC-PTP, IL-24, gp130, C-myc, LILRB4, BMP-2, MIA, CD34, CD63, CD9, CD81, IFNab R2, Glypican 2, MSP R, DSCAM, Matriptase, KIR2DL3, CD30, Siglec-10, CLEC-1, TPP1, Ubiquitin+1, ANGPTL4, TWEAK R, Nidogen-1, CD2, Kallikrein 1, TSLP R, LAMP1, TROY, VCAM-1, Siglec-11, S100A1, PARI, Thyroid Peroxidase, Aminopeptidase P2, IL-1 RI, ADAM9, OSM R beta, Thrombospondin-2, SMPD1, B2M, MFRP, LRP-6, ST3GAL1, NCAM-1 (CD56), Granzyme B, Adiponectin, IL-22BP, TPST2, PD-ECGF, LH, LEDGF, Cyr61, ULBP-3, IFNb, THSD1, FGF-23, LAMA4, Adipsin, AIF, SorCS2, SULT2A1, CD39L2, Insulin R, HIF-1 alpha, OX40 Ligand, Pax3, UCH-L3, cMASP3, Langerin, Desmin, SOX9, ST6GAL1, MEP1B, CD99-L2, Plexin A4, Semaphorin 4D, ROBO2, PDX-1, APRIL, Neurturin, Kremen-2, EMMPRIN, Activin RIB, Neuroligin 2, Epiregulin, CA5A, MMP-12, GALNT2, CEACAM-5, VEGF R1, DSPG3, SorCS1, Matrilin-2, sFRP-3, p53, EphB3, NCKI, Semaphorin 7A, NKp80, Prolactin, Cystatin B, Sirtuin 1, FGF-16, FGF R5, NQO-1, Semaphorin 6D, FGF-3, GATA-4, VAP-A, CHST2, Pappalysin-2, Syndecan-3, Jagged 1, AKR1C4, Olfactomedin-2, Osteoadherin, NKp44, Thyroglobulin, IL-21R, Chemerin, EphA1, CD48, MICB, FGF-5, TRANCE, CES2, ULBP-1, Integrin alpha 5, VAMP-2, FLRG, Ret Midkine, CD73, TRACP, proGRP, Granzyme H, PRX2, p27, Siglec-6, Dectin-1, CD51, Notch-1, Calreticulin, DR3, DCTN1, CDC25B, Osteoactivin, ACE, CA125, HAO-1, PSMA1, FCRLB, BMP-9, CRIM1, LIF, SPINK1, EphB6, RGM-B, HS3ST1, ROR1, CMG-2, 4-1BB Ligand, LICAM-2, p63, Cathepsin V, Testican 2, Glypican 5, CD6, Siglec-2, Legumain, PRELP, CES1, TAZ, NSE, TECK, HTRA2, HIF-1 beta, TAFA1, Podocalyxin, RalA, CRELD2, GRAP2, SP-D, BID, GFR alpha-2, Notch-3, VEGF R3, DLL4, TGFb2, LIGHT, XIAP, ST8SIA1, Cathepsin L, 6Ckine, MIS RII, Kallikrein 5, TGM3, FCAR, Contactin-2, CD83, IL-1 R3, SALM4, GBA3, ROBO4, OSCAR, VEGF, IGSF3, Biglycan, Neudesin, ILT4, uPAR, Axl, WIF-1, IL-7 R alpha, GPR56, CEACAM-3, MCEMPI, FABP2, Plexin B3, MEPE, Activin RIIA, ANG-2, Cochlin, Presenilin 1, NPTXR, SLAM, COMT, SPHKI, RBP4, Nectin-1, GUSB, Nidogen-2, IL-17F, SR-AI, TAFA2, N-Cadherin, IL-17B, IL-17 RC, MIP-3b, Cystatin C, Cystatin D, AMSH, FcERI, CLECIOA, HGF R, ANG-1, Prolactin R, FGF-20, CD28, Nogo-A, HSD17B1, IL-19, Enteropeptidase, Cathepsin E, TSLP, TCN2, GDF-15, Epimorphin, GRK5, PD-1, Serpin A4, ADAM23, NOV, Galectin-2, Neurexin 3 beta, TLR3, Sirtuin 2, Numb, IL-28 R alpha, IL-33, Lin28, FCRL1, KLF4, NKp30, Lymphotactin, Cystatin SN, JAM-A, Calreticulin-2, ErbB4, BMP-8, IL-27 Ra, Fas, IL-4 Ra, Kallikrein 14, Matrilin-3, Olig2, Kallikrein 12, CA13, IL-9, Nectin-3, MPIF-1, Cystatin S, ADA, IL-2 Rb, GFR alpha-1, Smad4, ICAM-1, MEF2C, TREM-1, L-Selectin, Hepsin, CD42b, MCSF, RANK, CHST4, CA8, FCRL3, ASAH2, CF XIV, PYY, HGF, I-TAC, Semaphorin 4C, SorCS3, Tie-1, IL-31 RA, Arginase 1, POGLUT1, IL-1ra, Podoplanin, TIM-3, CREG, CD300f, uPA, EphA2, LRRTM4, LIMPII, Tenascin R, CPE, PECAM-1, DNAM-1, DKK-1, OPG, CPB1, TSH, MMP-2, Siglec-9, ICAM-3, Cystatin SA, Galectin-4, Pepsinogen II, Desmoglein-3, Nectin-4, SCF, Serpin A5, PTH, FGF-19, MSP, IL-28A, FGF-12, METAP2, ASAHL, EDIL3, NTAL, EGF R, TAFA5, Galectin-9, vWF-A2, TACE, Activin RIIB, Cathepsin S, LDL R, BMPR-IA, OX40, IL-13 R2, B7-H4, MMP-13, ANGPTL7, TRAIL R4, IGSF4B, Sirtuin 5, PEAR1, SH2D1A, Cerberus 1, GDF-11, Nrf2, TROP-2, NUDT5, ROR2, EphB4, Glypican 1, LAP(TGFb1), Gas6, Contactin-1, IL-27, UNC5H4, ICAM-2, MBL, HS3ST3B1, RCOR1, IL-10 Rb, XEDAR, IL-22, PILR-alpha, NRGi-b1, FABP4, RGM-A, RELT, TrkC, C5a, SREC-I, Nestin, TPO, ErbB3, Kirrel3, FLRT1, Galectin-3, CXCL16, JAM-B, DR6, Nogo Receptor, TLR4, VEGF R2, Tie-2, IL-15 R, Caspr2, LTbR, LAMP, ALCAM, GLP-1, NG2, IL-22 R alpha 1, AMIGO2, HCC-1, TFPI-2, ULBP-2, Desmoglein 2, Aggrecan, Syntaxin 4, VAMP-1, Nectin-2, FGF-21, Flt-3, GFAP, TIM-1, Inhibin A, Cadherin-4, PlGF-2, Neurogranin, HE4, IL-23 R, Galectin-7, GALNT3, GITR L, CD14, R-Spondin 2, CK19, Cardiotrophin-1, TREML1, HAPLN1, CD27, ANG-4, Siglec-7, CD155, VEGF-C, TNF RII, PGRP-S, SDF-1a, PDGF-AB, GPVI, CD40, SCF R, Thrombospondin-5, IL-1 RII, Neuropilin-2, Cadherin-13, E-Selectin, GITR, WISP-1, Renin, AgRP, MDL-1, ROBO3, RANTES, Endocan, Granulysin, hCGb, Mesothelin, TLR1, TRAIL, MOG, DDR1, NGF R, TRAIL R3, Trypsin 3, ARSB, LIF R alpha, BAFF R, CD157, Granzyme A, 2B4, ESAM, IL-1 R4, CXCL14, IL-31, SIRP alpha, Uromodulin, CTRC, CEACAM-1, TARC, MIP-3a, SDF-1b, NKp46, MCP-3, IL-32 alpha, TGFb3 FOLR2, CD58, IL-23, CD36, TNFb, Shh-N, Ficolin-1, Reg4, ILT2, Mer, TREM-2, Flt-3L, CD5, IL-6, CD229, Insulin, Syntaxin 6, GRO, Bcl-w, Lipocalin-2, PDGF-AA, IL-2 Ra, Angiogenin, LYVE-1, CD4, RAGE, CDNF, Brevican, NAP-2, PU.1, EDAR, ADAMTS13, Kynureninase, PTH1R, IFN-gamma R1, CrkL, B7-1, PARC, Draxin, VE-Cadherin, Procalcitonin, SOX15, Kallikrein 11, BCMA, Dectin-2, EpCAM, HCC-4, TGFa, IP-10, BLAME, CILP-1, PIGF, LOX-1, MCP-2, Resistin, HVEM, ENPP-7, Syndecan-4, IL-2 Rg, MICA, Dopa Decarboxylase, NPDC-1, MCP-4, EG-VEGF, Glycoprotein V, Semaphorin 4G, IL-12p40, PSA-total, IL-15, MAP1D, Clq, TNF4, Dtk, Endoglin, ENA-78, Reg3A, MIP-1b, FGF-17, IL-6R, IL-8, Galectin-8, CA4, Cystatin E M, FUT8, B7-H3, GCP-2, CD40L, MDC, 4-1BB, HO-1, SOST, S100A13, Kallikrein 7, and IL-13.

In some embodiments, the composition comprises one or more biomolecules that can selectively bind to a microbial antigen (such as a viral, bacterial, fungal, or parasitic antigen), block its function and/or enzymatically process the protein so it is detectable by the host immune system to then activate virus immune response to disable the virus' ability to infect cells. For example, the biomolecule can bind to a a viral antigen from a virus selected from the group consisting of Herpes Simplex virus-1 (such as, for example, glycoprotein D and/or glycoprotein G), Herpes Simplex virus-2 (such as, for example, glycoprotein D and/or glycoprotein G), Varicella-Zoster virus (such as, for example, glycoprotein E), Epstein-Barr virus (such as, for example the EBV glycoprotein), Cytomegalovirus (such as, for example the CMV glycoprotein), Human Herpes virus-6, Variola virus, Vesicular stomatitis virus, Hepatitis A virus, Hepatitis B virus (including, but not limited to the hepatitis B virus surface antigen), Hepatitis C virus (such as, for example, the Hepatitis C E1, E2, or E3 proteins), Hepatitis D virus, Hepatitis E virus, Rhinovirus, Coronavirus (including, but not limited to spike or envelope proteins from avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2 (including, but not limited to the B1.351 variant, B.1.1.7 variant, and P.1 variant), or MERS-CoV), Influenza virus A (such as, for example the hemagglutinin (HA) protein including the HA1 and HA2 protein and including trimeric HA), Influenza virus B (such as, for example the hemagglutinin (HA) protein including the HA1 and HA2 protein and including trimeric HA), Measles virus (such as, for example the hemagglutinin protein), Polyomavirus, Human Papilomavirus, Respiratory syncytial virus (such as, for example the RSV G protein), Adenovirus, Coxsackie virus, Dengue virus (such as, for example capsid protein, envelope protein, and/or premembrane/membrane protein), Mumps virus, Poliovirus, Rabies virus (including, but not limited to the Rabies glycoprotein), Rous sarcoma virus, Reovirus, Yellow fever virus, Zika virus (such as, for example capsid protein, envelope protein, and/or premembrane/membrane protein), Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A (including, but not limited to viral protein 4 and viral protein 7), Rotavirus B (including, but not limited to viral protein 4 and viral protein 7), Rotavirus C (including, but not limited to viral protein 4 and viral protein 7), Sindbis virus, Simian Immunodeficiency virus, Human T-cell Leukemia virus type-1, Hantavirus, Rubella virus, Simian Immunodeficiency virus, Human Immunodeficiency virus type-1 (such as, for example, glycoprotein (gp), envelope protein (Env), or gag protein), and Human Immunodeficiency virus type-2. For example, the presence of furin protein in the invention may enable enzymatic processing of shed viral particle spike protein to the upright immune-detectable conformation, improving immune response to degrade extracellular viral particles. Additionally, for example, the composition can comprise a ferritin protein content effective to increase IL-10 secretion by immune regulatory cells to inhibit hyperactive immune cell actions, collectively referred to as a cytokine storm or the protein PAI-1 that can block production of plasmin to inhibit the “bradykinin storm.” The biomolecule of the composition can also comprise thrombomodulin, which can suppress micro-blood clotting frequency, reducing pathogenic clotting, and reduce thrombotic emboli; and other protein components that inhibit NETosis (neutrophil induced nucleic acid-protein networks intended to capture pathogenic invading species, for example, viruses, and bacteria within the vasculature)

Extracellular vesicles (EV) are small membrane bound spheres containing proteins and RNA (of which exosomes are a subset). Exosomes are small (e.g., 20-150 nm) diameter lipid bilayer vesicles secreted by cells to enable paracrine communication. Other EV populations are derived directly from the plasma membrane or are formed during apoptosis (apoptotic bodies). Recently, miRNA sequences homologous with the sequence of SARS-CoV-2 RNA were compared and a list of miRNAs were identified that appear able to bind to the viral sequence in the 3′ untranslated region, which has a low mutation rate. Therefore, the miRNA should be effective in their action against multiple variants of the virus. Accordingly, in some embodiments, the biomolecule of the composition comprises a micro RNA content that may bind to RNA sequences of a microbe and block translation from or activate degradation of the microbial RNA sequence. The composition thereby may reduce microbial replication rate (including, but not limited to viral replication rate) and reduce host cell death. For example, disclosed herein are compositions comprising a therapeutically effective amount of an MSC secretome (such as, for example, including, but not limited to MSC growth factor, MSC exosome, MSC extracts and/or extracellular vesicle comprising compositions) and one or more biomolecules comprise a miRNA is selected from the group of miRNA comprising hsa-let-7a-5p, hsa-let-7b-5p, hsa-let-7c-5p, hsa-let-7d-3p, hsa-let-7e-5p, hsa-let-7g-5p, hsa-let-7i, hsa-let-7i-5p, hsa-miR-100-5p, hsa-miR-103a-3p, hsa-miR-106a-5p, hsa-miR-106b-5p, hsa-mir-10b, hsa-miR-10b-5p, hsa-mir-1246, hsa-miR-1246, hsa-miR-125a-5p, hsa-miR-125b-5p, hsa-miR-130a-3p, hsa-mir-130b, hsa-miR-130b-3p, hsa-miR-132-3p, hsa-miR-136-5p, hsa-miR-138-5p, hsa-miR-139-5p, hsa-mir-140, hsa-miR-140-3p, hsa-miR-145-5p, hsa-mir-146a, hsa-miR-146a-5p, hsa-miR-148a-3p, hsa-miR-152-3p, hsa-miR-15a-5p, hsa-miR-15b-5p, hsa-mir-16-1, hsa-mir-16-2, hsa-miR-16-5p, hsa-miR-17-5p, hsa-miR-181a-5p, hsa-miR-191-5p, hsa-miR-193a-5p, hsa-miR-193b-3p, hsa-miR-197-3p, hsa-miR-199a-3p, hsa-miR-199a-5p, hsa-miR-199b-5p, hsa-miR-19a-3p, hsa-miR-19b-3p, hsa-miR-20a-5p, hsa-mir-203a, hsa-miR-203a-3p, hsa-miR-214-3p, hsa-mir-21, hsa-miR-21-3p, hsa-miR-21-5p, hsa-mir-221, hsa-miR-221-3p, hsa-mir-222, hsa-miR-222-3p, hsa-miR-22-3p, hsa-miR-23a-3p, hsa-miR-23b-3p, hsa-mir-24-1, hsa-mir-24-2, hsa-miR-24-3p, hsa-mir-25, hsa-miR-25-3p, hsa-miR-26a-5p, hsa-miR-27a-3p, hsa-mir-27b, hsa-miR-27b-3p, hsa-miR-29a-3p, hsa-miR-29c-3p, hsa-miR-30a-5p, hsa-miR-30a-5p, hsa-miR-30b-5p, hsa-miR-30c-5p, hsa-mir-30d, hsa-miR-30d-5p, hsa-mir-30e, hsa-miR-30e-5p, hsa-miR-31-3p, hsa-miR-31-5p, hsa-miR-320a, hsa-miR-342-3p, hsa-miR-345-5p, hsa-miR-34a-5p, hsa-miR-361-5p, hsa-miR-376a-3p, hsa-miR-376c-3p, hsa-miR-423-3p, hsa-miR-423-5p, hsa-miR-424-5p, hsa-miR-484, hsa-mir-486-1, hsa-mir-486-2, hsa-miR-486-5p, hsa-miR-570-3p, hsa-miR-574-3p, hsa-miR-663a, hsa-miR-874-3p, hsa-mir-92a-1, hsa-mir-92a-2, hsa-miR-92a-3p, hsa-miR-92b-3p, hsa-mir-93, hsa-miR-93-5p, hsa-miR-940, hsa-miR-99a-5p, and hsa-miR-99b-5p.

In some embodiments, the composition comprises miRNA (such as hsa-miR-19a-3p, hsa-miR-19b-3p) that can be effective to inhibit translation of Bradykinin receptor 2, which is critical for activation of bradykinin signaling responsible for severe vascular response to coronavirus infection.

In some embodiments, the composition comprises microRNA that can inhibit translation of Kallikrein B1 (such as hsa-miR-24-3p) and other Kallikrein proteins involved in proteolytic digestion of the bradykinin precursor protein to generate bradykinin peptide.

In some embodiments, the biomolecule of the composition comprises microRNA content that can inhibit translation of cellular proteins involved in enabling virus fusion to the cell membrane using the angiotensin-converting enzyme 2 (ACE 2) receptor protein or by blocking activity of proteins activated through the process of the virus binding to the ACE 2 receptor protein. For example, the transmembrane protease, serine 2 (TMPRSS2) enzyme can be inhibited. TMPRSS2 is required to enable SARS-CoV-2 spike protein to interact with the ACE 2 Receptor and initiate membrane fusion. Exemplary microRNA content may include human miRNA sequences hsa-let-7a-5p, hsa-let-7b-5p, hsa-let-7c-5p, hsa-let-7g-5p, hsa-let-7i-5p, hsa-miR-214-3p, and hsa-miR-27a-3p, which all have binding sites in mRNA for TMPRSS2.

In some embodiments, the biomolecule of the composition comprises microRNA that can inhibit proteins of the bradykinin pathway. For example, the microRNA sequences hsa-let-7a-5p, hsa-let-7b-5p, hsa-let-7c-5p, hsa-let-7g-5p, hsa-let-7i-5p, hsa-let-7i, hsa-miR-106a-5p, hsa-miR-106b-5p, hsa-miR-15a-5p, hsa-miR-15b-5p, hsa-miR-16-5p, hsa-miR-17-5p, hsa-miR-20a-5p, hsa-miR-23a-3p, hsa-miR-23b-3p, hsa-mir-24-1, hsa-mir-24-2, hsa-miR-25-3p, hsa-miR-92a-3p, hsa-miR-92b-3p, and hsa-miR-93-5p may bind and prevent translation of hyaluronan synthase 2, thereby preventing formation of Hyaluronic acid complexes in the lungs. Hyaluronic acid complexes block oxygen exchange in the alveoli.

In some embodiments, the biomolecule of the composition comprises a microRNA content, a protein content, or a combination thereof that can inhibit a cytokine storm. The composition may inhibit the cytokine storm by (i) inhibiting translation of cytokine proteins by binding mRNA sequences for those proteins, (ii) sterically hindering ligand/receptor interactions, (iii) enzymatically altering ligands or receptors to inhibit their pro-inflammatory actions, or (iv) activating inhibitory proteins, lipids or RNA sequences that inhibit the proinflammatory cytokines such as, but not limited to, IL-1beta, IL-6, TNF-alpha, GM-CSF, M-CSF. For example, human miRNA sequences hsa-let-7a-5p, hsa-let-7b-5p, hsa-let-7c-5p, hsa-let-7g-5p, hsa-let-7i-5p, and hsa-miR-547-3p are able to bind IL-6 mRNA, while hsa-miR-130a-3p and hsa-miR-181a-5p can inhibit translation of TNF-alpha.

It is understood and herein contemplated that the MSC secretome comprises exosomes and growth factors. The growth factors and exosomes can be allogenic or autogenic. The growth factors and exosomes can be derived from any cell in the human body, such as from ectodermal cells, endodermal cells, or mesodermal cells. For example, the MSC secretomes may comprise mesenchymal stem cell (MSC) derived growth factors, MSC derived exosomes, or both MSC derived growth factors and exosomes. In some embodiments, the method further comprises adding at least one additive with the exosomes and growth factors. Specifically, MSCs under appropriate wound healing conditions may produce suitable therapeutic agents, such as exosomes and growth factors, that can provide therapy for inflammatory lung diseases. In one aspect, disclosed herein are compositions, wherein the MSC secretome composition further comprises prostaglandin E2 (PGE2), transforming growth factor β1 (TGF-β1), hepatocyte growth factor (HGF), stromal cell derived factor-1 (SDF-1), nitric oxide, indoleamine 2,3-dioxygenase, interleukin-4 (IL-4), IL-6, interleukin-10 (IL-1β), IL-1 receptor antagonist and soluble TNF-α receptor, insulin-like growth factors, fibroblast growth factors (FGF) 1-23 (especially, FGF1 and FGF2), bone morphogenetic proteins (BMPs) 1-15, epidermal growth factor (EGF), transforming growth factor-α (TGF-α) macrophage-stimulating protein (MSP), platelet derived growth factor (PLGF), vascular endothelial growth factor (VEGF), macrophage colony stimulating factor (M-CSF), insulin, granulocyte colony stimulating factor (G-CSF), granulocyte macrophage colony stimulating factor (GM-CSF estrogen, and/or thyroid hormones.

As noted above, the composition comprises proteins and microRNAs, some of which may be embedded in or surrounded by a lipid membrane to create vesicles in the size range of about >20 nm to about 200 nm in size. The number of vesicles within the invention may range between about 1 million to about 100 billion vesicles per mL when suspended or about 10 million to about 1 trillion when formulated as a lyophilized powder.

### 1. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism such as, for example, a metered-dose inhaler, a dry powder inhaler, a nebulizer, a vaporization device, or the like. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis have been reviewed (Brown and Greene, DNA and Cell Biology 10:6, 399-409 (1991)).

### A) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution, and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### C. Mesenchymal Stem Cells

As noted throughout, the treatment compositions disclosed herein can utilize MSC secretomes and/or growth factors derived from mesenchymal stem cells (MSCs). While allogenic cellular MSC IV infusion treatments have been widely pursued, there are numerous safety and regulatory concerns surrounding allogenic cellular preparations. The inherent problems with IV infusions of living MSCs include the trapping of the cells in the lungs, causing the cells to die within 24 hours. The cellular debris from this cell death is transported to the liver and spleen, where it is disposed. Current autogenous treatments from bone marrow concentrate only deliver a few thousand MSCs. While allogenic expanded MSC IV infusions can deliver hundreds of millions of living MSCs, they get trapped in the lungs and die. The long-term effect of introducing the foreign DNA into the recipient is unclear and questions have arisen on whether introducing the large amount of foreign DNA could be carcinogenic.

Existing autogenous and allogeneic MSCs contained within bone marrow, bone marrow concentrate, synovia-derived mesenchymal stem cells (MSCs), or adipose-derived stromal vascular fraction (SVF) or various post-natal products from umbilical cord, placenta or amnion, expanded MSC cultures are currently being used to treat wounds, orthopedic pathology, and spine pathology. To avoid recognition by the immune system, undifferentiated MSCs express low to medium levels of human leukocyte antigen (HLA) Class I and low levels of HLA Class II. This property gives donor MSCs a so-called ‘stealth’ ability to go undetected by a host immune system in allogeneic therapies. However, Class I antigen is present at detectable levels and Class II antigen expression can be induced by INF-γ. Several cases of allogeneic MSC rejection and chronic immune responses have been reported in animal studies and human clinical trials. This entire problem could be avoided by using only the mesenchymal stem cell secretomes, including, but not limited to growth factors, proteins, peptides, glycosaminoglycans, proteoglycans, chemokines, cytokines, extracts, extracellular vesicles, and/or exosomes collected from the conditioned growth media. An acellular versus a cellular MSC product

In fact, prior to the present disclosure an active MSC growth factor product that can be used for these applications has not been developed. Thus, in one aspect, disclosed herein are MSC secretome compositions (including, but not limited to MSC growth factor, MSC exosome, MSC extracts and/or extracellular vesicle comprising compositions) for use in the treatment, inhibition, decrease, reduction, amelioration, and/or prevention of a microbial infection, symptoms of said infection, or secondary conditions (such as, for example, microbial initiated cytokine storm, microbial infection initiated bradykinin storm, and/or microbial infection induced acute respiratory distress syndrome), said treatment compositions comprising (i) a growth factor powdered additive comprising a mesenchymal stem cell (MSC) derived preparation (including, but not limited to a MSC secretome composition) and (ii) a pharmaceutically acceptable carrier.

As noted above, MSC are multipotent cells that have the ability to differentiate into a multitude of cell types including myocytes, chondrocytes, adipocytes, and osteoblasts. Typically, these cells can be found in the placenta, umbilical cord blood, adipose tissue, bone marrow, or amniotic fluid, including perivascular tissue. As used herein, “MSC” refers to non-terminally differentiated cells including but not limited to multipotential stem cell, multipotential stromal cell, stromal vascular cells, pericytes, perivascular cells, stromal cells, pluripotent cells, multipotent cells, adipose-derived fibroblast-like cells, adipose-derived stromal vascular fraction, adipose-derived MSC, bone marrow-derived fibroblast-like cells, bone marrow-derived stromal vascular fraction, bone marrow-derived MSC, tissue-derived fibroblast-like cells, adult stem cells, adult stromal cells, keratinocytes, and/or melanocytes.

It has been long recognized that MSC, in addition to their differentiation potential, have the immunomodulatory abilities resulting in the expression of many different cytokines and growth factors. As used herein, a “MSC preparation” or “MSC secretome composition” refers to a composition comprising MSC growth factors, MSC exosomes, extracellular vesicles, extracellular vesicle isolate product (EVIP), or acellular extracts of MSCs and/or MSC lysates obtained from human MSCs, fibroblast-like cells, and non-human animal MSCs including, but not limited to MSCs from horses, cows, pigs, sheep, non-human primates, dogs, cats, rabbits, rats, and mice. In embodiments, the MSCs may be derived from the patient to which the composition will be applied (autologous) or derived from another individual (allogeneic). The MSCs may be culture expanded to collect the conditioned media or to increase the quantity of cells for the lysate or used freshly prior to incorporation into the composition of the present disclosure.

The MSC secretome compositions (including, but not limited to MSC growth factor, MSC exosome, MSC extracts and/or extracellular vesicle comprising compositions) may comprise about 0.00001 to about 20 wt. %, such as from about 0.01 to about 10 wt. %, of a mesenchymal stem cell (MSC) extract, MSC exosome, or MSC growth factor preparation. The MSC preparation may comprise either MSC conditioned media or MSC lysate from cell culture expanded MSCs. In some embodiments, the composition may further comprise from about 0.01 to about 10 wt. % of a cell-free medium conditioned by growth of MSCs or MSC lineage cells, wherein the cells are cultured under normal hyperoxic culturing conditions or under artificial wound healing conditions.

As disclosed herein the MSCs used to produce the disclosed MSC additives (including growth factor secretome composition either frozen or powdered additives) can be selectively stimulated to produce MSC growth factors, secretomes, cytokines, chemokines, mesenchymal stem cell proteins, peptides, glycosaminoglycans, extracellular matrix (ECM), proteoglycans, secretomes, and exosomes. The growth factors and exosomes may be derived from any cell in the human body, such as from ectodermal cells, endodermal cells, or mesodermal cells. As used herein, MSC growth factors include but are not limited to prostaglandin E2 (PGE2), transforming growth factor β1 (TGF-β1), hepatocyte growth factor (HGF), stromal cell derived factor-1 (SDF-1), nitric oxide, indoleamine 2,3-dioxygenase, interleukin-4 (IL-4), IL-6, interleukin-10 (IL-1β), IL-1 receptor antagonist and soluble TNF-α receptor, insulin-like growth factors, fibroblast growth factors (FGF) 1-23 (especially, FGF1 and FGF2), bone morphogenetic proteins (BMPs) 1-15, epidermal growth factor (EGF), transforming growth factor-α (TGF-α) macrophage-stimulating protein (MSP), platelet derived growth factor (PLGF), vascular endothelial growth factor (VEGF), macrophage colony stimulating factor (M-CSF), insulin, granulocyte colony stimulating factor (G-CSF), granulocyte macrophage colony stimulating factor (GM-CSF), as well as hormones including estrogen, and thyroid hormones.

As mentioned above, culturing the MSCs may occur under normal hyperoxic culturing conditions or under wound healing hypoxic conditions. Hyperoxic conditions may comprise of approximately 21% oxygen with or without serum supplements, while hypoxic conditions may comprise about 1% to about 5% oxygen with inflammatory cytokines, angiogenic factors, reduced serum, reduced glucose or these elements in various combinations. The combined reduced nutrient and metabolite environment may trigger the cultured cells to produce wound healing and anti-inflammatory ECM proteins and growth factors to direct tissue healing. Direct tissue healing likely is in the form of new ECM proteins, such as collagen and glycosaminoglycans (GAGs), as well as growth factors and cytokines. In one aspect, the MSC preparation (such as, for example, a MSC secretome composition) comprises MSC growth factors, MSC exosomes, and/or cellular extracts of MSCs or MSC lysates obtained from MSCs cultured under standard hyperoxic culturing conditions (for example, 21% oxygen) or MSCs cultured under artificial wound healing conditions (such as, for example, 0.1% to about 5% oxygen in the presence of inflammatory cytokines, angiogenic factors, and reduced glucose).

As disclosed herein artificial wound healing conditions simulate growth conditions in real wounds where there is a reduction in nutrient supply and reduction of waste removal that is usually caused by a disruption in local blood circulation. This creates a harsh environment for cells until new blood vessels are created and blood circulation is restored. Accordingly, artificial wound healing conditions used to culture MSCs can include one or more of the following growth conditions reduction in glucose availability, reduction in oxygen tension, reduction in pH, and increased temperature.

In one aspect, the glucose availability can be reduced relative to normal control. Modified culture media to reduce glucose, but not damage the cells can be between 0 and 50% reduction in glucose, more preferably between about 5% and 40% reduction in glucose. For example, MSC artificial wound healing culture conditions can comprise glucose reduction of about 1, 2, 3, 4, 5, 6, 7, 8 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50% such as a glucose reduction from about 5% to about 15%, from about 10% to about 20%, from about 15% to about 25%, from about 20% to about 30%, or from about 25% to about 35%.

In one aspect, oxygen tension can be reduced to oxygen levels to hypoxic conditions. Normal atmospheric oxygen is approximately 21% and any reduction is considered hypoxic. Thus, in one aspect, MSCs can be cultured at between 0.0% and 2.9% oxygen, from about 0.1% to about 0.5% oxygen, from about 0.1% to about 2.0%, from about 0.1% to about 5.0% oxygen, from about 0.5% to 5.0%, from about 1.0% to about 10% oxygen, about 5.0% to about 10.0% oxygen; and from about 10.0% to about 15.0% under artificial wound healing conditions. Preferably during MSC would healing culture conditions oxygen tension is between about 0.5% and 20.5% oxygen, such as, for example, 0, 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.7, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 10.5, 11, 11.5, 12, 12.5, 13, 13.5, 14, 14.5, 15, 15.5, 16, 16.5, 17, 17.5, 18, 18.5, 19, 19.5, 20, or 20.5% oxygen.

The pH can also be reduced under artificial wound healing conditions. Physiologic pH is maintained very tightly and is usually very close to a neutral pH=7.2±0.2 (7.0-7.4). However, in a wound the acidic environment can have a pH=6.2±0.2 (i.e., a pH from 6.0 to about 6.4). Thus, under artificial wound healing culture conditions, pH can be from about 6.0 to about 7.4, for example, from 6.0 to about 6.4, from about 6.2 to about 6.4, from about 6.2 to about 6.6, from about 6.4 to about 6.6, from about 6.4 to about 6.8, or from about 6.6 to about 7.0, such as 6.0, 6.1, 6.2, 6.3, 6.4, 6.5, 6.6, 6.7, 6.8, 6.9, 7.0, 7.1, 7.2, 7.3 or 7.4.

Under artificial wound healing culture conditions, the temperature of the culture environment may be raised to simulate temperature increases at the site of a wound. Physiologic homeostasis temperature is maintained at 37° C. (98.6° F.). A slight increase or decrease can cause significant changes to cellular metabolism. By increasing the temperature above 37° C. to any temperature up to about 40° C. (104° F.) can create an “feverous” environment. Thus, in on aspect, the artificial wound healing culture conditions for the MSCs can comprise from about 35° C. to about 39° C., from about 35° C. to about 36° C., from about 36° C. to about 37° C., from about 37° C. to about 38° C., from about 38° C. to about 39° C., from about 39° C. to about 40° C. In one aspect, the temperature of the artificial wound healing culture can be 35.0, 35.1, 35.2, 35.3, 36.4, 35.5, 35.6, 35.7, 35.8, 35.9, 36.0, 36.1, 36.2, 36.3, 36.4, 36.5, 36.6, 36.7, 36.8, 36.9, 37.0, 37.1, 37.2, 37.3, 37.4, 37.5, 37.6, 37.7, 37.8, 37.9, 38.0, 38.1, 38.2, 38.3, 38.4, 38.5, 38.6, 38.7, 38.8, 38.9, 39.0, 39.1, 39.2, 39.3, 39.4, 39.5, 39.6, 39.7, 39.8, 39.9, or 40.0° C.

In one aspect, the MSC secretome compositions (including, but not limited to MSC growth factor, MSC exosome, MSC extracts and/or extracellular vesicle comprising compositions) can further comprise a protective coating (such as, for example, a cryoprotectant oligosaccharide and a protein solution) to reduce degradation of the growth factors. It is understood and herein contemplated that the protective coating can be engineered as a polymer. “Polymer” refers to a relatively high molecular weight organic compound, natural or synthetic, whose structure can be represented by a repeated small unit, the monomer. Non-limiting examples of polymers include polyethylene, rubber, cellulose. Synthetic polymers are typically formed by addition or condensation polymerization of monomers. The term “copolymer” refers to a polymer formed from two or more different repeating units (monomer residues). By way of example and without limitation, a copolymer can be an alternating copolymer, a random copolymer, a block copolymer, or a graft copolymer. It is also contemplated that, in certain aspects, various block segments of a block copolymer can themselves comprise copolymers. The term “polymer” encompasses all forms of polymers including, but not limited to, natural polymers, synthetic polymers, homopolymers, heteropolymers or copolymers, addition polymers, etc. In one aspect, the gel matrix can comprise copolymers, block copolymers, diblock copolymers, and/or triblock copolymers.

In one aspect, the protective coating can comprise a biocompatible polymer. In one aspect, biocompatible polymer can be crosslinked. Such polymers can also serve to slowly release the adipose browning agent and/or fat modulating agent into tissue. As used herein biocompatible polymers include, but are not limited to polysaccharides; hydrophilic polypeptides; poly(amino acids) such as poly-L-glutamic acid (PGS), gamma-polyglutamic acid, poly-L-aspartic acid, poly-L-serine, or poly-L-lysine; polyalkylene glycols and polyalkylene oxides such as polyethylene glycol (PEG), polypropylene glycol (PPG), and poly(ethylene oxide) (PEO); poly(oxyethylated polyol); poly(olefinic alcohol); polyvinylpyrrolidone); poly(hydroxyalkylmethacrylamide); poly(hydroxyalkylmethacrylate); poly(saccharides); poly(hydroxy acids); poly(vinyl alcohol), polyhydroxyacids such as poly(lactic acid), poly (gly colic acid), and poly (lactic acid-co-glycolic acids); polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), as well as copolymers thereof. Biocompatible polymers can also include polyamides, polycarbonates, polyalkylenes, polyalkylene glycols, polyalkylene oxides, polyalkylene terepthalates, polyvinyl alcohols (PVA), methacrylate PVA(m-PVA), polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly (methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), poly(vinyl acetate, poly vinyl chloride polystyrene and polyvinylpryrrolidone, derivatives thereof, linear and branched copolymers and block copolymers thereof, and blends thereof. Exemplary biodegradable polymers include polyesters, poly(ortho esters), poly(ethylene amines), poly(caprolactones), poly(hydroxybutyrates), poly(hydroxyvalerates), polyanhydrides, poly(acrylic acids), polyglycolides, poly(urethanes), polycarbonates, polyphosphate esters, polyphospliazenes, derivatives thereof, linear and branched copolymers and block copolymers thereof, and blends thereof.

In some embodiments the protective coating comprises carbohydrate construction of monosaccharides as well as carbohydrate polymers such as disaccharides or polysaccharides including but not limited to non-reducing poly or disaccharides as well as any combination thereof. Examples of carbohydrates that can be used in the protective coating comprise Glucose, Aldoses (D-Allose, D-Altrose, D-Mannose, etc.), Glucopyranose, Pentahydroxyhexanal, α-D-Glucopyranosyl-D-glucose, α-D-Glucopyranosyl-dihydrate, Polymer of β-D-Glycopyranosyl units, β-D-Fructofuranosyl α-D-glucopyranoside (anhydrous/dihydrate), β-D-Galactopyranosyl-D-glucose, α-D-Glucopyranosyl-α-D-glucopyranoside (anhydrous/dihydrate), Galactose, Pentoses (Ribose, xylose, lyxose), Dextrose, Dodecacarbon monodecahydrate, Fructose, Sucrose, Lactose, Maltose, Trehalose, Agarose, D-galactosyl-β-(1-4)-anhydro-L-galactosyl, Cellulose, Polymer of β-D-Glycopyranosyl units, and Starch, as well as, Polyhydric alcohols, Polyalcohols, Alditols, Erythritol, Glycitols, Glycerol, Xylitol, and Sorbitol.

In some embodiments the protective coating contains biocompatible and/or biodegradable polyesters or polyanhydrides such as poly(lactic acid), poly(glycolic acid), and poly(lactic-co-glycolic acid). The particles can contain one more of the following polyesters: homopolymers including glycolic acid units, referred to herein as “PGA”, and lactic acid units, such as poly-L-lactic acid, poly-D-lactic acid, poly-D,L-lactic acid, poly-L-lactide, poly-D-lactide, and poly-D,L-lactide5 collectively referred to herein as “PLA”, and caprolactone units, such as poly(e-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; and polyacrylates, and derivatives thereof. Exemplary polymers also include copolymers of polyethylene glycol (PEG) and the aforementioned polyesters, such as various forms of PLGA-PEG or PLA-PEG copolymers, collectively referred to herein as “PEGylated polymers”. In certain embodiments, the PEG region can be covalently associated with polymer to yield “PEGylated polymers” by a cleavable linker. In one aspect, the polymer comprises at least 60, 65, 70, 75, 80, 85, 89, 90, 91, 92, 93, 94, 95, 96, 97, 98, or 99 percent acetal pendant groups.

The triblock copolymers disclosed herein comprise a core polymer such as, example, polyethylene glycol (PEG), polyvinyl acetate, polyvinyl alcohol, polyvinyl pyrrolidone (PVP), polyethyleneoxide (PEO), poly(vinyl pyrrolidone-co-vinyl acetate), polymethacrylates, polyoxyethylene alkyl ethers, polyoxyethylene castor oils, polycaprolactam, polylactic acid, polyglycolic acid, poly(lactic-glycolic) acid, poly(lactic co-glycolic) acid (PLGA), cellulose derivatives, such as hydroxymethylcellulose, hydroxypropylcellulose and the like.

Examples of diblock copolymers that can be used in the protective coatings disclosed herein comprise a polymer such as, example, polyethylene glycol (PEG), polyvinyl acetate, polyvinyl alcohol (PVA), polyvinyl pyrrolidone (PVP), polyethyleneoxide (PEO), poly(vinyl pyrrolidone-co-vinyl acetate), polymethacrylates, polyoxyethylene alkyl ethers, polyoxyethylene castor oils, polycaprolactam, polylactic acid, polyglycolic acid, poly(lactic-glycolic) acid, poly(lactic co-glycolic) acid (PLGA).

In one aspect, the protective coating contains (i.e., the encapsulated, the encapsulated compositions can further comprise lecithin or hydrolyzed lecithin as a carrier or as encapsulation material. As used herein, lecithin and/or hydrolyzed lecithin coatings include coatings comprising phosphatidyl choline, phosphatidyl inositol, phosphatidyl ethanolamine, phosphatidylserine, and phosphatidic acid. Sources of the lecithin can be pnat or animal sources.

In one aspect, any of the polymers, monosaccharides, disaccharides, or polysaccharides used to form the protective coating formed by placing the MSC additive in an encapsulating solution can be at an appropriate concentration for form the protective coating. For example, polymers, monosaccharides, disaccharides, or polysaccharides can be at any concentration between 0.01 mM and 10.0 M concentration, for example, from about 0.01M to about 0.1M, from about 0.1 mM to about 1.0 M, from about 1.0 M to about 10.0 M. Exemplary concentrations include 0.01, 0.02, 0.03, 0.04, 0.05, 0.06, 0.07, 0.08, 0.09, 0.1, 0.2, 0.3, 0.4, 0.4, 0.6, 0.7, 0.8, 0.9, 1, 2, 3, 4, 5, 6, 7, 8, 9, 10, 20, 30, 40, 50, 60, 70, 80, 90, 100, 110, 120, 130, 140, 150, 160, 170, 180, 190, 200, 225, 250, 275, 300, 325, 350, 375, 400, 450, 500, 600, 700, 800, 900 mM, 1, 1.1, 1.2, 1.3, 1.4, 1.5, 1.6, 1.7, 1.8, 1.9, 2, 3, 4, 5, 6, 7, 8, 9, 10M.

In one aspect, the MSC secretome compositions (including, but not limited to MSC growth factor, MSC exosome, MSC extracts and/or extracellular vesicle comprising compositions) disclosed herein may comprise any known ingredients typically found pharmaceutical fields such as agents for combating free radicals; bactericides; sequestering agents; preservatives; basifying or acidifying agents; fragrances; surfactants; fillers; natural products or extracts of natural product, such as aloe or green tea extract; vitamins; or coloring materials. Other ingredients that may be combined with the powder may include an antioxidant, which can be selected from a variety of antioxidants. Suitable antioxidants include vitamins, such as Vitamin C (L-Ascorbate, Ascorbate-2 Phosphate magnesium salt, Ascorbyl Palmitate, Tetrahexyldecyl Ascorbate), Vitamin E (Tocotrienol), Vitamin A (retinol, retinal, retinoic acid, provitamin A carotenoids, such as beta-carotene), N-acetyl glucosamine, or other derivatives of glucosamine. Other ingredients may include at least one essential fatty acid, such as Q-3, Q-6, and Q-9 polyunsaturated fatty acids, such as linoleic acid (LA), gamma-linoleic acid (GLA), alpha-linoleic acid (ALA), dihomo-y-linolenic acid (DGLA), arachidonic acid (ARA), and others. The fatty acids may be derived from various sources including evening primrose oil, black currant oil, borage oil, or GLA modified safflower seeds. Other ingredients may include a platelet rich fibrin matrix, at least one ingredient to support ECM production and production of hyaluronic acid, such as N-acetyl glucosamine or other derivatives of glucosamine, ultra-low molecular weight (ULMW) hyaluronic acid, chondroitin sulfate, or keratin sulfate.

Producing the MSC secretome compositions can comprise culturing MSCs collected from a donor to create a cultured media under culturing conditions selected from the group consisting of normal hyperoxic culturing conditions and wound healing hypoxic conditions including reduced oxygen and nutrition; stimulating the cultured cells to selectively secrete desired anti-inflammatory proteins, peptides, glycosaminoglycans, proteoglycans exosomes, and secretomes by adjusting the cell growth conditions; collecting, combining the conglomerate mixture with an encapsulation solution, and freezing the conglomerate mixture, wherein the conglomerate mixture comprises exosomes, peptides, proteins, cytokines, growth factors, extracellular matrix (ECM), proteoglycans, glycosaminoglycans; and chemokines selected from the group consisting of human MSCs, animal MSCs, multipotential stromal cells, fibroblasts, and fibroblast cells; combining the conglomerate mixture with an encapsulation solution, such as oligosaccharides, like a trehalose solution or protein solution and freezing the mixture; and lyophilizing or freeze-drying the frozen mixture, creating a dry powder. Alternatively, the MSCs may be lysed to collect all of the MSCs from the culture process, creating an extracted lysate; concentrating the extracted lysate and combining the extracted lysate with an encapsulation solution, such as oligosaccharides like a trehalose solution or protein solution and freezing the mixture; and lyophilizing or freeze-drying the frozen mixture, creating a dry powder. The powder contains a highly concentrated collection of analgesic MSC secretomes and exosomes and extracellular matrix components that are specific to anti-inflammation.

The method may also include filter-sterilizing, concentrating, freezing, or freeze drying the MSC conditioned culture medium. Additionally, the MSC culture medium may be combined with a cryoprotectant prior to freezing.

There are various methods for lysing the MSCs. Lysing may be achieved by the addition of a hypotonic solution or repeated freeze-thaw processes to disrupt the cell membranes. Moreover, the cells may be lysed while attached to the culture surface or in suspension. The cells may also be enzymatically released and/or lysed by mechanical homogenization.

Stimulating the MSC to selectively secrete the desired anti-inflammatory proteins, peptides, glycosaminoglycans, proteoglycans, exosomes and secretomes may be achieved by adjusting the cell growth conditions, such as cell confluency, culture media supplements, nutritional supplements, oxygen levels, length of culture in those conditions, cell passage number or combinations of those, and the like.

### D. Methods of Treating Microbial Infections and/or Symptoms Thereof

In one aspect, it is understood and herein contemplated that the disclosed comprising a therapeutically effective amount of a MSC secretome (such as, for example, including, but not limited to MSC growth factor, MSC exosome, MSC extracts and/or extracellular vesicle comprising compositions) and one or more biomolecules (such as, for example, a peptide, polypeptide, protein, siRNA, shRNA, and/or microRNA (miRNA)) that selectively bind to one or more microbial immunogens, or inhibit the ability of a microbe to inhabit a host, or inhibit, decrease, reduce, ameliorate, and/or prevent one or more secondary conditions caused by a microbial infection (such as, for example, comprise a peptide, polypeptide, protein, siRNA, shRNA, microRNA (miRNA) can act concurrently and synergistically to directly affect viral infection and/or replication and/or act concurrently and synergistically to regulate downstream inflammation and vascular cell related pathologies in response to microbial infection). Accordingly, disclosed herein are methods of treating, decreasing, inhibiting, reducing, ameliorating and/or preventing a microbial infection (such as, for example, a viral, bacterial, fungal, or parasitic infection) or symptoms thereof (including, but not limited to microbial induced cytokine storm, microbial initiated bradykinin storm, and/or acute respiratory distress syndrome) in a subject comprising administering to a subject any of the compositions disclosed herein. For example, disclosed herein are methods of treating, decreasing, inhibiting, reducing, ameliorating and/or preventing a microbial infection (such as, for example, a viral, bacterial, fungal, or parasitic infection) or symptoms thereof (including, but not limited to microbial induced cytokine storm, microbial initiated bradykinin storm, and/or acute respiratory distress syndrome) in a subject comprising administering to a subject a composition comprising a therapeutically effective amount of a MSC secretome (such as, for example, including, but not limited to MSC growth factor, MSC exosome, MSC extracts and/or extracellular vesicle comprising compositions) and one or more biomolecules (such as, for example, a peptide, polypeptide, protein, siRNA, shRNA, and/or microRNA (miRNA)) that selectively bind to one or more microbial immunogens, or inhibit the ability of a microbe to inhabit a host, or inhibit, decrease, reduce, ameliorate, and/or prevent one or more secondary conditions caused by a microbial infection (such as, for example, comprise a peptide, polypeptide, protein, siRNA, shRNA, microRNA (miRNA).

As noted above, the composition for use in the disclosed methods can comprises bone marrow-derived mesenchymal lineage cells that adhere to culture plastic and may differentiate in culture into multiple mature cell fates including but not limited to adipocyte, osteoblast and chondrocyte fates and/or secreted extracellular vesicles that contain a core protein composition that includes any combination of core composition proteins, selected from the group consisting of Ferritin, NUP85, LAMP2, GPR115, Serpin F1, OPN, PAI-1, DAPP1, Cathepsin B, Semaphorin 6C, PDGF R alpha, Sortilin, Serpin B6, Dkk-3, Thrombomodulin, PF4, MIF, Periostin, Furin, TIMP-1, Decorin, PCKT, CD99, CD63, CD9, CD81, Transferrin, DcR3, Lumican, TIMP-2, SLITRK5, FAP, Artemin, DPPII, cIAP-1, Pentraxin 3, Visfatin, Neprilysin, Albumin, Galectin-1, UNC5H3, IL-20 R beta, SREC-II, JAM-C, TNF RI, htPAPP-A, eNOS, MSP R, TPP1, LAMP1, B2M, NCAM-1, HIF-1 alpha, ST6GAL1, CD99-L2, Plexin A4, EMMPRIN, p53, Semaphorin 7A, NKp80, Cystatin B, Osteoadherin, Midkine, Calreticulin, Osteoactivin, Legumain, TAZ, Cathepsin L, RBP4, Serpin A4, JAM-A, MCSF, LIMPII, OPG, IL-22, Galectin-3, MOG, Trypsin 3, SIRP alpha, and Syndecan-4, and at least one protein selected from the group consisting of: Ferritin, IGFBP-4 IL-1 R6 GSTM1, NUP85, LAMP2, MeprinA, IL-1 F10, bIG-H3, GPR115, TGFbT, Ephrin-A4, CD109, Serpin F1, IGFBP-6, HS3ST4, Aminopeptidase LRAP, OPN, PAI-1, DAPP1, GDF-9, Cathepsin B, IGFBP-2, Semaphorin 6C, IGF-2, PDGF R alpha, Sortilin, Serpin B6, Dkk-3, CNTF, TSP-1, GM-CSF Ra, Thrombomodulin, Endoglycan, IGFBP-3, RGM-C, PF4, MIF, TGM4, Periostin, Furin, TIMP-1, PAPP-A, Decorin, PCK1, Arylsulfatase A, CD99, CA2, PRDX4, Transferrin, DcR3, GP73, LAIR2, ULBP-4, Lumican, TIMP-2, TFPI, SOX2, SLITRK5, FAP, Spinesin, ENPP-2, CD97, CTACK, Integrin alpha 1, EXTL3, IL-18 BPa, PD-L2, PSMA, IL-20 Ra, Glyoxalase II, Trypsin 1, IGF-2R, ADAMTSL-1, Erythropoietin, Plexin D1, DNMT3A, BCL-2, CL-P1, Ephrin-B3, FABP6, CHI3L1, FCRL5, TFF3, Artemin, DPPII, cIAP-1, PDGF Rb, Pentraxin 3, Angiotensinogen, Follistatin, CF VII, Persephin, TRAIL RI, THAP11, CD200, CLEC-2, AMIGO, IGFBP-5, PON1, SOX7, GALNT10, Visfatin, Progranulin, PCSK2, GKN1, IL-18, Neprilysin, Stabilin-2, IL-17 RD, Albumin, Follistatin-like 1, MMP-10, FKBP51, LRRC4, Pref-1, Galectin-1, Troponin C, UNC5H3, FLRT2, CD314, Semaphorin 6B, Netrin-4, CD27 Ligand, IL-20 R beta, Semaphorin 6A, TSK, Cytokeratin-8, CHST3, Mcl-1, DPPIV, SREC-II, Norrin, JAM-C, Bel-10, Wnt-4, LSECtin, Kell, TNF RI, PTP1B, htPAPP-A, IDO, PDGF-CC, Galanin, Activin A, TLR2, SCCA2, FABP1, eNOS, SHP-1, ICOS, ClqTNF9, MMP-1, TC-PTP, IL-24, gp130, C-myc, LILRB4, BMP-2, MIA, CD34, CD63, CD9, CD81, IFNab R2, Glypican 2, MSP R, DSCAM, Matriptase, KIR2DL3, CD30, Siglec-10, CLEC-1, TPP1, Ubiquitin+1, ANGPTL4, TWEAK R, Nidogen-1, CD2, Kallikrein 1, TSLP R, LAMP1, TROY, VCAM-1, Siglec-11, S100A1, PARI, Thyroid Peroxidase, Aminopeptidase P2, IL-1 RI, ADAM9, OSM R beta, Thrombospondin-2, SMPD1, B2M, MFRP, LRP-6, ST3GAL1, NCAM-1 (CD56), Granzyme B, Adiponectin, IL-22BP, TPST2, PD-ECGF, LH, LEDGF, Cyr61, ULBP-3, IFNb, THSD1, FGF-23, LAMA4, Adipsin, AIF, SorCS2, SULT2A1, CD39L2, Insulin R, HIF-1 alpha, OX40 Ligand, Pax3, UCH-L3, cMASP3, Langerin, Desmin, SOX9, ST6GAL1, MEP1B, CD99-L2, Plexin A4, Semaphorin 4D, ROBO2, PDX-1, APRIL, Neurturin, Kremen-2, EMMPRIN, Activin RIB, Neuroligin 2, Epiregulin, CA5A, MMP-12, GALNT2, CEACAM-5, VEGF R1, DSPG3, SorCS1, Matrilin-2, sFRP-3, p53, EphB3, NCKI, Semaphorin 7A, NKp80, Prolactin, Cystatin B, Sirtuin 1, FGF-16, FGF R5, NQO-1, Semaphorin 6D, FGF-3, GATA-4, VAP-A, CHST2, Pappalysin-2, Syndecan-3, Jagged 1, AKR1C4, Olfactomedin-2, Osteoadherin, NKp44, Thyroglobulin, IL-21R, Chemerin, EphA1, CD48, MICB, FGF-5, TRANCE, CES2, ULBP-1, Integrin alpha 5, VAMP-2, FLRG, Ret Midkine, CD73, TRACP, proGRP, Granzyme H, PRX2, p27, Siglec-6, Dectin-1, CD51, Notch-1, Calreticulin, DR3, DCTN1, CDC25B, Osteoactivin, ACE, CA125, HAO-1, PSMA1, FCRLB, BMP-9, CRIM1, LIF, SPINK1, EphB6, RGM-B, HS3ST1, ROR1, CMG-2, 4-1BB Ligand, L1CAM-2, p63, Cathepsin V, Testican 2, Glypican 5, CD6, Siglec-2, Legumain, PRELP, CES1, TAZ, NSE, TECK, HTRA2, HIF-1 beta, TAFA1, Podocalyxin, RalA, CRELD2, GRAP2, SP-D, BID, GFR alpha-2, Notch-3, VEGF R3, DLL4, TGFb2, LIGHT, XIAP, ST8SIA1, Cathepsin L, 6Ckine, MIS RII, Kallikrein 5, TGM3, FCAR, Contactin-2, CD83, IL-1 R3, SALM4, GBA3, ROBO4, OSCAR, VEGF, IGSF3, Biglycan, Neudesin, ILT4, uPAR, Axl, WIF-1, IL-7 R alpha, GPR56, CEACAM-3, MCEMPI, FABP2, Plexin B3, MEPE, Activin RIIA, ANG-2, Cochlin, Presenilin 1, NPTXR, SLAM, COMT, SPHKI, RBP4, Nectin-1, GUSB, Nidogen-2, IL-17F, SR-AI, TAFA2, N-Cadherin, IL-17B, IL-17 RC, MIP-3b, Cystatin C, Cystatin D, AMSH, FcERI, CLECIOA, HGF R, ANG-1, Prolactin R, FGF-20, CD28, Nogo-A, HSD17B1, IL-19, Enteropeptidase, Cathepsin E, TSLP, TCN2, GDF-15, Epimorphin, GRK5, PD-1, Serpin A4, ADAM23, NOV, Galectin-2, Neurexin 3 beta, TLR3, Sirtuin 2, Numb, IL-28 R alpha, IL-33, Lin28, FCRL1, KLF4, NKp30, Lymphotactin, Cystatin SN, JAM-A, Calreticulin-2, ErbB4, BMP-8, IL-27 Ra, Fas, IL-4 Ra, Kallikrein 14, Matrilin-3, Olig2, Kallikrein 12, CA13, IL-9, Nectin-3, MPIF-1, Cystatin S, ADA, IL-2 Rb, GFR alpha-1, Smad4, ICAM-1, MEF2C, TREM-1, L-Selectin, Hepsin, CD42b, MCSF, RANK, CHST4, CA8, FCRL3, ASAH2, CF XIV, PYY, HGF, I-TAC, Semaphorin 4C, SorCS3, Tie-1, IL-31 RA, Arginase 1, POGLUT1, IL-1ra, Podoplanin, TIM-3, CREG, CD300f, uPA, EphA2, LRRTM4, LIMPII, Tenascin R, CPE, PECAM-1, DNAM-1, DKK-1, OPG, CPB1, TSH, MMP-2, Siglec-9, ICAM-3, Cystatin SA, Galectin-4, Pepsinogen II, Desmoglein-3, Nectin-4, SCF, Serpin A5, PTH, FGF-19, MSP, IL-28A, FGF-12, METAP2, ASAHL, EDIL3, NTAL, EGF R, TAFA5, Galectin-9, vWF-A2, TACE, Activin RIIB, Cathepsin S, LDL R, BMPR-IA, OX40, IL-13 R2, B7-H4, MMP-13, ANGPTL7, TRAIL R4, IGSF4B, Sirtuin 5, PEAR1, SH2D1A, Cerberus 1, GDF-11, Nrf2, TROP-2, NUDT5, ROR2, EphB4, Glypican 1, LAP(TGFb1), Gas6, Contactin-1, IL-27, UNC5H4, ICAM-2, MBL, HS3ST3B1, RCOR1, IL-10 Rb, XEDAR, IL-22, PILR-alpha, NRGi-b1, FABP4, RGM-A, RELT, TrkC, C5a, SREC-I, Nestin, TPO, ErbB3, Kirrel3, FLRT1, Galectin-3, CXCL16, JAM-B, DR6, Nogo Receptor, TLR4, VEGF R2, Tie-2, IL-15 R, Caspr2, LTbR, LAMP, ALCAM, GLP-1, NG2, IL-22 R alpha 1, AMIGO2, HCC-1, TFPI-2, ULBP-2, Desmoglein 2, Aggrecan, Syntaxin 4, VAMP-1, Nectin-2, FGF-21, Flt-3, GFAP, TIM-1, Inhibin A, Cadherin-4, PlGF-2, Neurogranin, HE4, IL-23 R, Galectin-7, GALNT3, GITR L, CD14, R-Spondin 2, CK19, Cardiotrophin-1, TREML1, HAPLN1, CD27, ANG-4, Siglec-7, CD155, VEGF-C, TNF RII, PGRP-S, SDF-1a, PDGF-AB, GPVI, CD40, SCF R, Thrombospondin-5, IL-1 RII, Neuropilin-2, Cadherin-13, E-Selectin, GITR, WISP-1, Renin, AgRP, MDL-1, ROBO3, RANTES, Endocan, Granulysin, hCGb, Mesothelin, TLR1, TRAIL, MOG, DDR1, NGF R, TRAIL R3, Trypsin 3, ARSB, LIF R alpha, BAFF R, CD157, Granzyme A, 2B4, ESAM, IL-1 R4, CXCL14, IL-31, SIRP alpha, Uromodulin, CTRC, CEACAM-1, TARC, MIP-3a, SDF-1b, NKp46, MCP-3, IL-32 alpha, TGFb3 FOLR2, CD58, IL-23, CD36, TNFb, Shh-N, Ficolin-1, Reg4, ILT2, Mer, TREM-2, Flt-3L, CD5, IL-6, CD229, Insulin, Syntaxin 6, GRO, Bcl-w, Lipocalin-2, PDGF-AA, IL-2 Ra, Angiogenin, LYVE-1, CD4, RAGE, CDNF, Brevican, NAP-2, PU.1, EDAR, ADAMTSi3, Kynureninase, PTH1R, IFN-gamma R1, CrkL, B7-1, PARC, Draxin, VE-Cadherin, Procalcitonin, SOX15, Kallikrein 11, BCMA, Dectin-2, EpCAM, HCC-4, TGFa, IP-10, BLAME, CILP-1, PIGF, LOX-1, MCP-2, Resistin, HVEM, ENPP-7, Syndecan-4, IL-2 Rg, MICA, Dopa Decarboxylase, NPDC-1, MCP-4, EG-VEGF, Glycoprotein V, Semaphorin 4G, IL-12p40, PSA-total, IL-15, MAP1D, C1q, TNF4, Dtk, Endoglin, ENA-78, Reg3A, MIP-1b, FGF-17, IL-6R, IL-8, Galectin-8, CA4, Cystatin E M, FUT8, B7-H3, GCP-2, CD40L, MDC, 4-1BB, HO-1, SOST, S100A13, Kallikrein 7, and IL-13.

In some embodiments, the composition for use in the disclosed methods comprises one or more biomolecules that can selectively bind to a microbial antigen (such as a viral, bacterial, fungal, or parasitic antigen), block its function and/or enzymatically process the protein so it is detectable by the host immune system to then activate virus immune response to disable the virus' ability to infect cells. For example, the biomolecule can bind to a viral antigen from a virus selected from the group consisting of Herpes Simplex virus-1 (such as, for example, glycoprotein D and/or glycoprotein G), Herpes Simplex virus-2 (such as, for example, glycoprotein D and/or glycoprotein G), Varicella-Zoster virus (such as, for example, glycoprotein E), Epstein-Barr virus (such as, for example the EBV glycoprotein), Cytomegalovirus (such as, for example the CMV glycoprotein), Human Herpes virus-6, Variola virus, Vesicular stomatitis virus, Hepatitis A virus, Hepatitis B virus (including, but not limited to the hepatitis B virus surface antigen), Hepatitis C virus (such as, for example, the Hepatitis C E1, E2, or E3 proteins), Hepatitis D virus, Hepatitis E virus, Rhinovirus, Coronavirus (including, but not limited to spike or envelope proteins from avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2 (including, but not limited to the B1.351 variant, B.1.1.7 variant, and P.1 variant), or MERS-CoV), Influenza virus A (such as, for example the hemagglutinin (HA) protein including the HA1 and HA2 protein and including trimeric HA), Influenza virus B (such as, for example the hemagglutinin (HA) protein including the HA1 and HA2 protein and including trimeric HA), Measles virus (such as, for example the hemagglutinin protein), Polyomavirus, Human Papilomavirus, Respiratory syncytial virus (such as, for example the RSV G protein), Adenovirus, Coxsackie virus, Dengue virus (such as, for example capsid protein, envelope protein, and/or premembrane/membrane protein), Mumps virus, Poliovirus, Rabies virus (including, but not limited to the Rabies glycoprotein), Rous sarcoma virus, Reovirus, Yellow fever virus, Zika virus (such as, for example capsid protein, envelope protein, and/or premembrane/membrane protein), Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A (including, but not limited to viral protein 4 and viral protein 7), Rotavirus B (including, but not limited to viral protein 4 and viral protein 7), Rotavirus C (including, but not limited to viral protein 4 and viral protein 7), Sindbis virus, Simian Immunodeficiency virus, Human T-cell Leukemia virus type-1, Hantavirus, Rubella virus, Simian Immunodeficiency virus, Human Immunodeficiency virus type-1 (such as, for example, glycoprotein (gp), envelope protein (Env), or gag protein), and Human Immunodeficiency virus type-2. For example, the presence of furin protein in the invention may enable enzymatic processing of shed viral particle spike protein to the upright immune-detectable conformation, improving immune response to degrade extracellular viral particles. Additionally, for example, the composition can comprise a ferritin protein content effective to increase IL-10 secretion by immune regulatory cells to inhibit hyperactive immune cell actions, collectively referred to as a cytokine storm or the protein PAI-1 that can block production of plasmin to inhibit the “bradykinin storm.” The biomolecule of the composition can also comprise thrombomodulin, which can suppress micro-blood clotting frequency, reducing pathogenic clotting, and reduce thrombotic emboli; and other protein components that inhibit NETosis (neutrophil induced nucleic acid-protein networks intended to capture pathogenic invading species, for example, viruses, and bacteria within the vasculature)

In some embodiments, the biomolecule of the composition for use in the disclosed methods comprises a micro RNA content that may bind to RNA sequences of a microbe and block translation from or activate degradation of the microbial RNA sequence. The composition thereby may reduce microbial replication rate (including, but not limited to viral replication rate) and reduce host cell death. For example, disclosed herein are compositions comprising a therapeutically effective amount of an MSC secretome (such as, for example, including, but not limited to MSC growth factor, MSC exosome, MSC extracts and/or extracellular vesicle comprising compositions) and one or more biomolecules comprise a miRNA is selected from the group of miRNA comprising hsa-let-7a-5p, hsa-let-7b-5p, hsa-let-7c-5p, hsa-let-7d-3p, hsa-let-7e-5p, hsa-let-7g-5p, hsa-let-7i, hsa-let-7i-5p, hsa-miR-100-5p, hsa-miR-103a-3p, hsa-miR-106a-5p, hsa-miR-106b-5p, hsa-mir-10b, hsa-miR-10b-5p, hsa-mir-1246, hsa-miR-1246, hsa-miR-125a-5p, hsa-miR-125b-5p, hsa-miR-130a-3p, hsa-mir-130b, hsa-miR-130b-3p, hsa-miR-132-3p, hsa-miR-136-5p, hsa-miR-138-5p, hsa-miR-139-5p, hsa-mir-140, hsa-miR-140-3p, hsa-miR-145-5p, hsa-mir-146a, hsa-miR-146a-5p, hsa-miR-148a-3p, hsa-miR-152-3p, hsa-miR-15a-5p, hsa-miR-15b-5p, hsa-mir-16-1, hsa-mir-16-2, hsa-miR-16-5p, hsa-miR-17-5p, hsa-miR-181a-5p, hsa-miR-191-5p, hsa-miR-193a-5p, hsa-miR-193b-3p, hsa-miR-197-3p, hsa-miR-199a-3p, hsa-miR-199a-5p, hsa-miR-199b-5p, hsa-miR-19a-3p, hsa-miR-19b-3p, hsa-miR-20a-5p, hsa-mir-203a, hsa-miR-203a-3p, hsa-miR-214-3p, hsa-mir-21, hsa-miR-21-3p, hsa-miR-21-5p, hsa-mir-221, hsa-miR-221-3p, hsa-mir-222, hsa-miR-222-3p, hsa-miR-22-3p, hsa-miR-23a-3p, hsa-miR-23b-3p, hsa-mir-24-1, hsa-mir-24-2, hsa-miR-24-3p, hsa-mir-25, hsa-miR-25-3p, hsa-miR-26a-5p, hsa-miR-27a-3p, hsa-mir-27b, hsa-miR-27b-3p, hsa-miR-29a-3p, hsa-miR-29c-3p, hsa-miR-30a-5p, hsa-miR-30a-5p, hsa-miR-30b-5p, hsa-miR-30c-5p, hsa-mir-30d, hsa-miR-30d-5p, hsa-mir-30e, hsa-miR-30e-5p, hsa-miR-31-3p, hsa-miR-31-5p, hsa-miR-320a, hsa-miR-342-3p, hsa-miR-345-5p, hsa-miR-34a-5p, hsa-miR-361-5p, hsa-miR-376a-3p, hsa-miR-376c-3p, hsa-miR-423-3p, hsa-miR-423-5p, hsa-miR-424-5p, hsa-miR-484, hsa-mir-486-1, hsa-mir-486-2, hsa-miR-486-5p, hsa-miR-570-3p, hsa-miR-574-3p, hsa-miR-663a, hsa-miR-874-3p, hsa-mir-92a-1, hsa-mir-92a-2, hsa-miR-92a-3p, hsa-miR-92b-3p, hsa-mir-93, hsa-miR-93-5p, hsa-miR-940, hsa-miR-99a-5p, and hsa-miR-99b-5p. For example, a composition comprising miRNA (such as hsa-miR-19a-3p, hsa-miR-19b-3p) that can be effective to inhibit translation of Bradykinin receptor 2, which is critical for activation of bradykinin signaling responsible for severe vascular response to coronavirus infection. Similarly, wherein the composition comprises microRNA that can inhibit translation of Kallikrein B1 (such as hsa-miR-24-3p) and other Kallikrein proteins involved in proteolytic digestion of the bradykinin precursor protein to generate bradykinin peptide. Also, for example, wherein the biomolecule of the composition comprises microRNA content that can inhibit translation of cellular proteins involved in enabling virus fusion to the cell membrane using the angiotensin-converting enzyme 2 (ACE 2) receptor protein or by blocking activity of proteins activated through the process of the virus binding to the ACE 2 receptor protein. For example, the transmembrane protease, serine 2 (TMPRSS2) enzyme can be inhibited. TMPRSS2 is required to enable SARS-CoV-2 spike protein to interact with the ACE 2 Receptor and initiate membrane fusion. Exemplary microRNA content may include human miRNA sequences hsa-let-7a-5p, hsa-let-7b-5p, hsa-let-7c-5p, hsa-let-7g-5p, hsa-let-7i-5p, hsa-miR-214-3p, and hsa-miR-27a-3p, which all have binding sites in mRNA for TMPRSS2. Additionally, wherein the biomolecule of the composition comprises microRNA that can inhibit proteins of the bradykinin pathway. For example, the microRNA sequences hsa-let-7a-5p, hsa-let-7b-5p, hsa-let-7c-5p, hsa-let-7g-5p, hsa-let-7i-5p, hsa-let-7i, hsa-miR-106a-5p, hsa-miR-106b-5p, hsa-miR-15a-5p, hsa-miR-15b-5p, hsa-miR-16-5p, hsa-miR-17-5p, hsa-miR-20a-5p, hsa-miR-23a-3p, hsa-miR-23b-3p, hsa-mir-24-1, hsa-mir-24-2, hsa-miR-25-3p, hsa-miR-92a-3p, hsa-miR-92b-3p, and hsa-miR-93-5p may bind and prevent translation of hyaluronan synthase 2, thereby preventing formation of Hyaluronic acid complexes in the lungs. Hyaluronic acid complexes block oxygen exchange in the alveoli. In some embodiments of the methods disclosed herein, the biomolecule of the composition comprises a microRNA content, a protein content, or a combination thereof that can inhibit a cytokine storm. The composition may inhibit the cytokine storm by (i) inhibiting translation of cytokine proteins by binding mRNA sequences for those proteins, (ii) sterically hindering ligand/receptor interactions, (iii) enzymatically altering ligands or receptors to inhibit their pro-inflammatory actions, or (iv) activating inhibitory proteins, lipids or RNA sequences that inhibit the proinflammatory cytokines such as, but not limited to, IL-1beta, IL-6, TNF-alpha, GM-CSF, M-CSF. For example, human miRNA sequences hsa-let-7a-5p, hsa-let-7b-5p, hsa-let-7c-5p, hsa-let-7g-5p, hsa-let-7i-5p, and hsa-miR-547-3p are able to bind IL-6 mRNA, while hsa-miR-130a-3p and hsa-miR-181a-5p can inhibit translation of TNF-alpha.

It is understood and herein contemplated that the MSC secretome used in the compositions of the disclosed methods can comprises exosomes and growth factors. The growth factors and exosomes can be allogenic or autogenic. The growth factors and exosomes can be derived from any cell in the human body, such as from ectodermal cells, endodermal cells, or mesodermal cells. For example, the MSC secretomes may comprise mesenchymal stem cell (MSC) derived growth factors, MSC derived exosomes, or both MSC derived growth factors and exosomes. In some embodiments, the method further comprises adding at least one additive with the exosomes and growth factors. Specifically, MSCs under appropriate wound healing conditions may produce suitable therapeutic agents, such as exosomes and growth factors, that can provide therapy for inflammatory lung diseases. In one aspect, disclosed herein are compositions of any preceding aspect, wherein the MSC secretome composition further comprises prostaglandin E2 (PGE2), transforming growth factor β1 (TGF-β1), hepatocyte growth factor (HGF), stromal cell derived factor-1 (SDF-1), nitric oxide, indoleamine 2,3-dioxygenase, interleukin-4 (IL-4), IL-6, interleukin-10 (IL-1β), IL-1 receptor antagonist and soluble TNF-α receptor, insulin-like growth factors, fibroblast growth factors (FGF) 1-23 (especially, FGF1 and FGF2), bone morphogenetic proteins (BMPs) 1-15, epidermal growth factor (EGF), transforming growth factor-α (TGF-α) macrophage-stimulating protein (MSP), platelet derived growth factor (PLGF), vascular endothelial growth factor (VEGF), macrophage colony stimulating factor (M-CSF), insulin, granulocyte colony stimulating factor (G-CSF), granulocyte macrophage colony stimulating factor (GM-CSF estrogen, and/or thyroid hormones.

As noted above, the microbial infection can comprise a viral, bacterial, fungal, or parasitic infection. In one aspect, disclosed herein are methods of treating, decreasing, inhibiting, reducing, ameliorating and/or preventing a microbial infection or symptoms thereof of any preceding aspect, wherein the microbial infection comprises an infection from a virus selected from the group of viruses consisting of Herpes Simplex virus- 1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2, or MERS-CoV), Influenza virus A, Influenza virus B, Measles virus, Polyomavirus, Human Papillomavirus, Respiratory syncytial virus, Adenovirus, Coxsackie virus, Chikungunya virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

Also disclosed herein are methods of treating, decreasing, inhibiting, reducing, ameliorating and/or preventing a microbial infection or symptoms thereof of any preceding aspect, wherein the microbial infection comprises an infection from a bacteria selected from the group of bacteria consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Mycobacterium chimaera, Nocardia asteroides, other Nocardia species, Legionella pneumophila, other Legionella species, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella bumetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species, and Mycoplasma species. In one aspect the bacteria is not Bacillus anthracis.

In one aspect, also disclosed herein are methods of treating, decreasing, inhibiting, reducing, ameliorating and/or preventing a microbial infection or symptoms thereof of any preceding aspect, wherein the microbial infection comprises an infection from a fungus selected from the group of fungi consisting of Candida albicans, Cryptococcus neoformans, Histoplasma capsulatum, Aspergillus fumigatus, Coccidiodes immitis, Paracoccidioides brasiliensis, Blastomyces dermitidis, Pneumocystis carinii, Penicillium marneffi, and Altemaria altemata.

Also disclosed herein are methods of treating, decreasing, inhibiting, reducing, ameliorating and/or preventing a microbial infection or symptoms thereof of any preceding aspect, wherein the microbial infection comprises a parasitic infection with a parasite selected from the group of parasitic organisms consisting of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, and Entamoeba histolytica.

### E. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Experimental treatments have shown that the treatment method of the present disclosure has greatly improved the health in critical cases of COVID-19, resulting in all test patients avoiding use of ventilators or being removed from a ventilator within 72 hours.

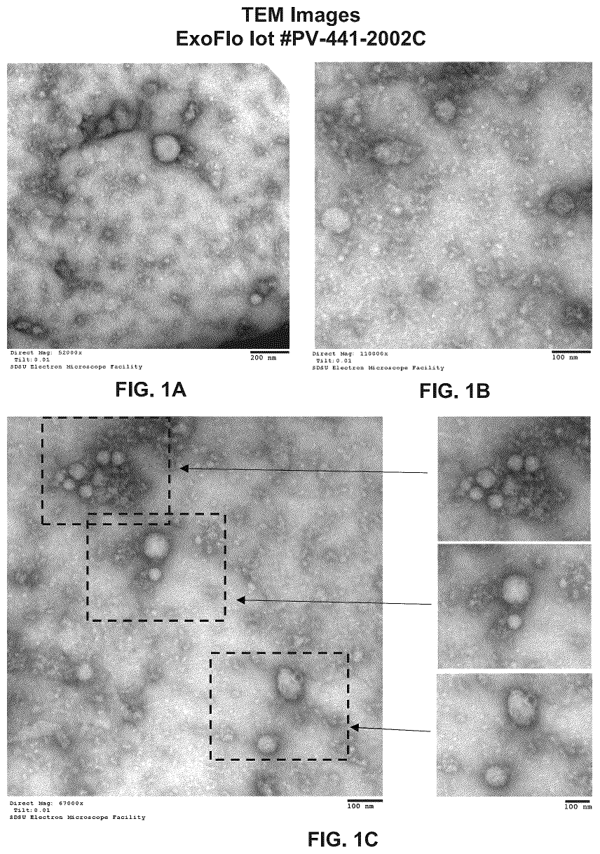

FIGS. 1A-1C show representative images captured by transmission electron microscopy (TEM) showing size distribution of the EVs present within an invention sample from a cyclic Guanosine Monophosphate (cGMP) manufactured lot #PV-441-2002C. FIGS. 1A, 1B, and 1C have scale bars of 200 nm, 100 nm, and 100 nm, respectively. Images 1, 2, and 3 are detail views of structures shown in FIG. 1C. Single Particle Interferometric Reflectance Imaging Sensor (SP-IRIS) is an imaging system used to capture image data of nanoparticles and transforms information into particle size, concentration, and identity. One such commercial system is the NANOVIEW ExoView™ system.

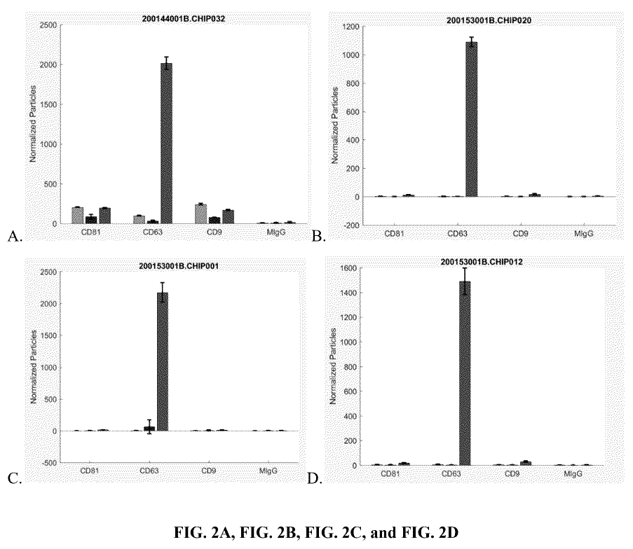

FIGS. 2A-2D shows CD81, CD63, and CD9 indicating captured exosomes. FIG. 2A is a chart that illustrates results of Lyophilized Test Lot 4411901C. FIG. 2B is a chart that illustrates results of pre-excipient Test Lot 441901D. FIG. 2C is a chart that illustrates results of pre-excipient Test Lot 441902A. FIG. 2D is a chart that illustrates results of pre-excipient Test Lot 441902B. All samples were stained with fluorescently labeled monoclonal antibodies. CD63 antibody (Ab) is indicated by red, CD81 Ab is indicated by green, and CD9 Ab is indicated by blue. FIG. 2 includes results for the following proteins. CD9 refers to a protein expressed in exosomes and plasma membrane EVs, a member of the tetraspanin protein family, associated with integrins. CD9 regulates sperm-egg interactions, platelet aggregation and activation, and cell adhesion. In myocytes, CD9 associates with CD81 and inhibits myotube formation during muscle regeneration. In monocytes/macrophages, CD9 associates with CD81 and integrins and prevents giant cell and osteoclast formation. CD63 refers to a protein expressed in exosomes, a member of the tetraspanin protein family. CD63 functions as the surface receptor for TIMP-1, activates cellular signaling cascades, promotes cell survival, and activates AKT and FAK/PTK2 pathways. CD81 refers to a protein expressed in exosomes, a member of the tetraspanin protein family. In myocytes, CD81 associates with CD9 and inhibits myotube formation during muscle regeneration. In monocytes/macrophages, CD81 associates with CD9 and integrins and prevents giant cell and osteoclast formation. CD81 is expressed in B-cells and is involved in CD19 receptor trafficking.

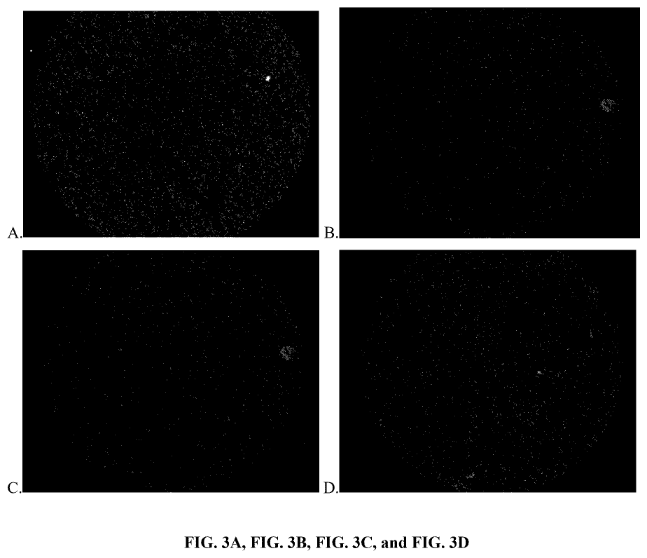

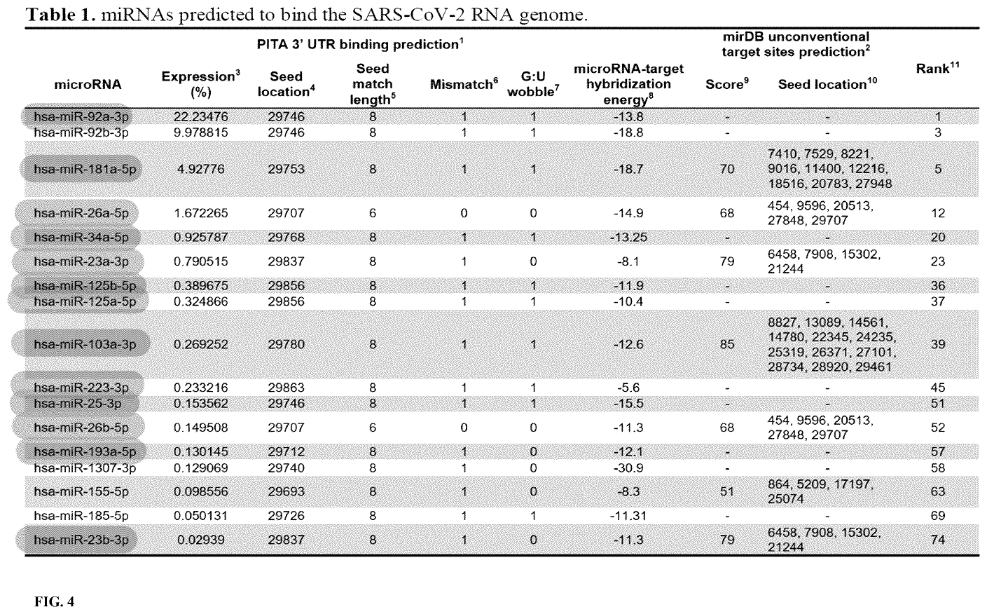

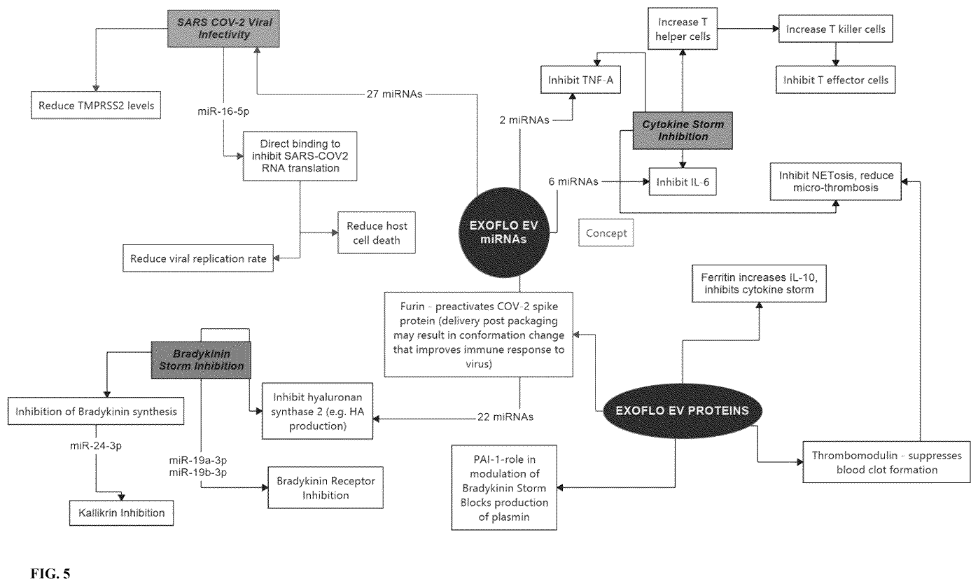

FIGS. 3A-3D are a set of microphotographs of CD63 Captured Exosomes. FIG. 3A illustrates lyophilized Test Lot 4411901C. FIG. 3B illustrates pre-excipient Test Lot 441901D. FIG. 3C illustrates pre-excipient Test Lot 441902A. FIG. 3D illustrates pre-excipient Test Lot 441902B. The image resolution limit for all images is 20 nm. All samples are stained with CD63 antibody (in red), CD81 Ab (in green), and CD9 Ab (in blue). Nanoparticle Tracking Analysis (NTA) is a method that allows visualizing and analyzing particles in suspension.

FIG. 4 reproduces a list of MSC secretome (also referred to under the trade name EXOFLO™) microRNA content identified as Table 1 in Park et al. Table 1 was highlighted to identify sequences found in EXOFLO™ that may directly bind to the SARS-CoV-2 virus RNA sequence. The Table 1 footnotes include the following notes. 1. Predictions of 3′ UTR binding were conducted with the PITA tool. 2. To predict miRNA binding to the 3′ UTR and region, analysis of the whole genome was also performed using miRDB. 3. Expression (%) indicates the level of each miRNA relative to the total miRNA levels, calculated miRNA CPM/total CPM*100. 4. Seed location where each miRNA is expected to bind the SARS-CoV-2 genome. 5. Seed match length. 6. Seed mismatch length. 7. Wobble number caused by G and U base pairing. 8. Thermodynamic energy required for binding. Lower energy indicates stronger binding prediction. The higher the score, the greater the likelihood of strong binding. 9. The prediction score from the miRDB method. According to miRDB, a predicted target with score >80 is most likely to be tightly bound. 10. Start site of the miRNA seed location matching to the RNA genome of SARS-CoV-2. 11. Rank of expression level within extracellular vesicles (EVs).

FIG. 5 is a schematic representation of multiple mechanisms of action discussed herein by which the invention may ameliorate coronavirus infection and subsequent symptoms.

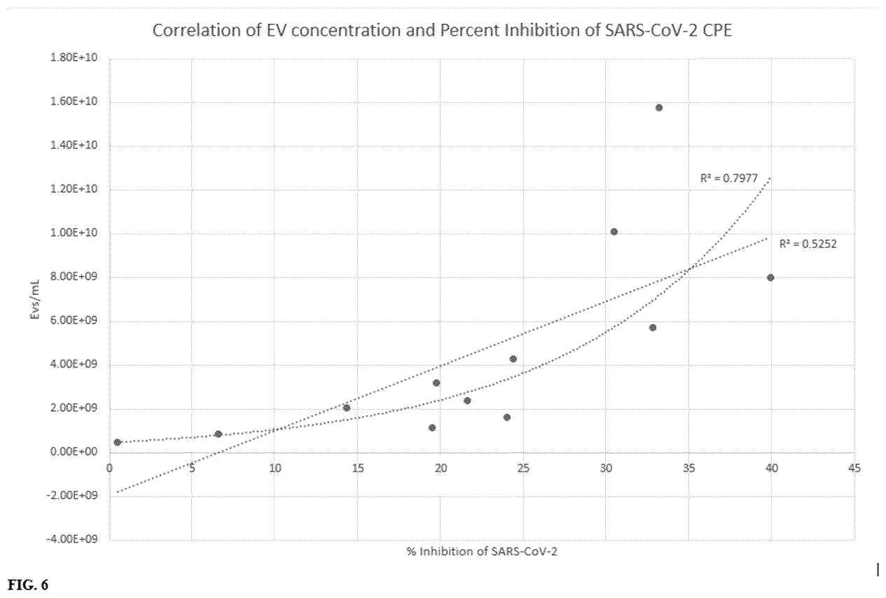

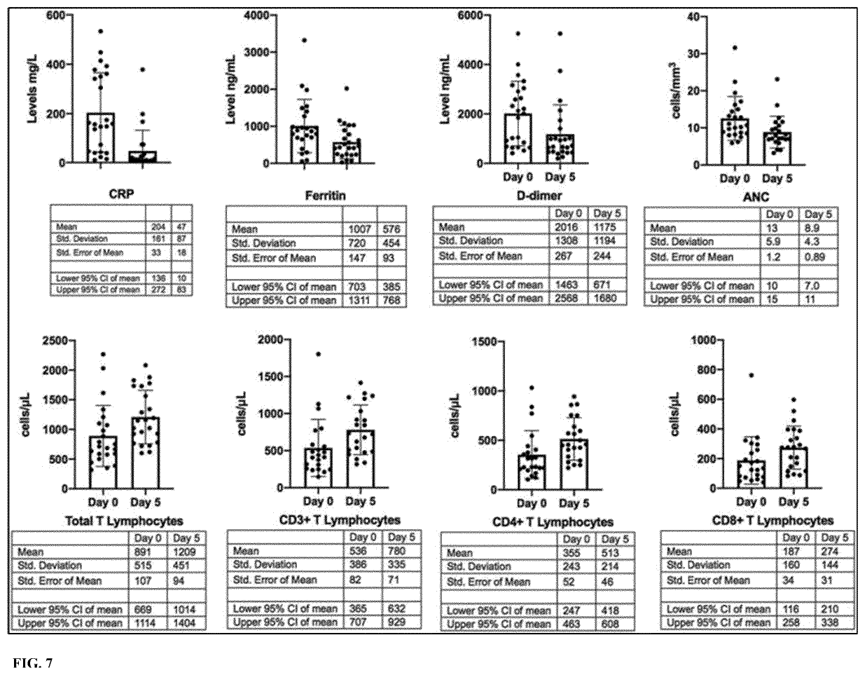

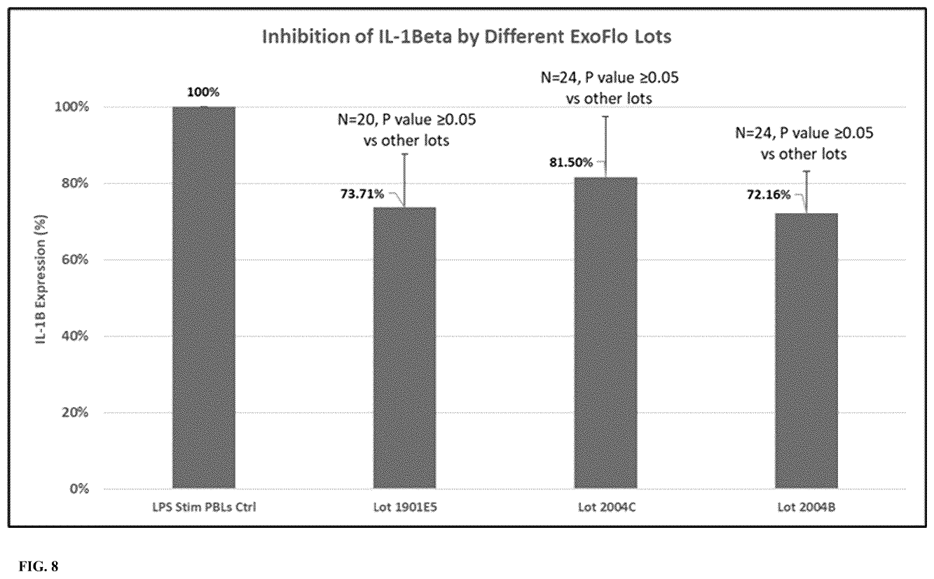

FIG. 6 is a graphic representation of viral toxicity inhibition (infectivity and replication) as a function of extracellular vesicle concentration, indicating a dose response relationship of the inventive composition on an in vitro live SARS CoV-2 virus in a “cyto-protective” assay. FIG. 6 includes broken lines indicating a linear regression and an exponential regression of the data.

FIG. 7 is a compilation of graphs demonstrating the effect of the invention when administered to patients with severe COVID-19 related ARDS. Biomarkers in clinical study are indicators of mechanism of action to reduce hyperinflammatory state (cytokine storm).

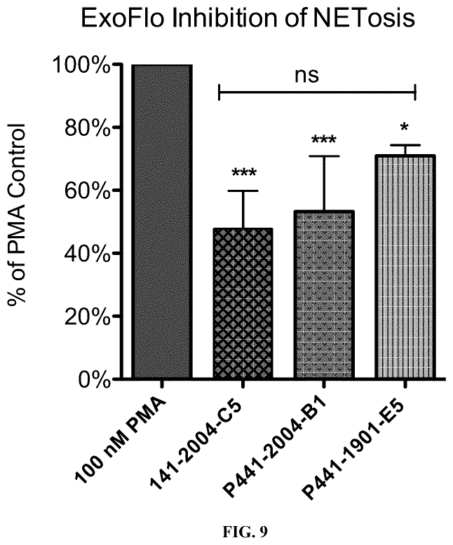

FIG. 8 is a graph illustrating one mechanism of action is to inhibit the master inflammation inducing cytokine, IL-1β. COVID-19 severity is linked to peripheral blood levels of IL-1β. Shown here is the capacity of the disclosed MSC secretome compositions (also referred to under the trade name EXOFLO™) to consistently regulate inflammation through inhibition of the proinflammatory cytokine IL-10 in LPS (lipopolysaccharides, or endotoxin) stimulated human peripheral blood mononuclear cells, an in vitro model used to simulation onset of hyperimmune response, also known as “cytokine storm.”

FIG. 9 illustrates that one mechanism of action is to regulate a hyper-active immune response by neutrophils by decreasing neutrophil extracellular trap formation (Netosis). Shown is the ability of the MSC secretomes (also referred to under the trade name EXOFLO™) to inhibit viral or bacterial induced NETosis through inhibition of neutrophil NET formation and reduce incidence of NET related micro-thrombi formation, for example, that associated with SARS-CoV-2 formation, or high levels of endotoxin associated with bacterial infection. Thus, reduction of NET formation is associated with a decrease in COVID-19 related disseminated intravascular coagulation (micro-clot formation).

What is claimed is:

1. A method for treating acute respiratory distress syndrome (ARDS), comprising administering to a subject in need thereof a composition comprising bone morphogenetic protein 1 (BMP-1), stem cell growth factor 1 (SCF 1), insulin-like growth factor 2 (IGF-2), TIMP-1 (tissue inhibitor of metalloproteinases 1), TIMP-2 (tissue inhibitor of metalloproteinases 2), transforming growth factor β1 (TGF-β1), CD63 antigen, ferritin protein, IGFBP-4 (Insulin-like growth factor binding protein-4), MIF (Macrophage migration inhibitory factor), macrophage colony stimulating factor (M-CSF), prostaglandin E2 (PGE2), hepatocyte growth factor (HGF), vascular endothelial growth factor (VEGF), stromal cell derived factor-1 (SDF-1), IL18BP (Interleukin-18 Binding Protein), indoleamine 2,3-dioxygenase, interleukin-4 (IL-4), interleukin-6 (IL-6), interleukin-10 (IL-10) interleukin 1 receptor inhibitor (IL-1 RI), soluble TNF-a receptor, fibroblast growth factor 1 (FGF-1), FGF-3, FGF-5, FGF-12, FGF-16, FGF-17, FGF-19, FGF-20, FGF-21, FGF-23, BMP-2, BMP-8, BMP-9, transforming growth factor-a (TGF-a), macrophage-stimulating protein (MSP), granulocyte macrophage colony stimulating factor (GM-CSF), PAI-1, or thrombomodulin, or any combination of two or more thereof.

2. The method of claim 1, wherein the composition (i) increases interleukin-10 (IL-10) secretion by immune regulatory cells to inhibit hyperactive immune cell action; (ii) blocks plasmin production; (iii) suppress micro-blood clotting frequency; (iv) reduces thrombotic emboli; (v) inhibits a pathogen-induced cell death or NETosis; or (vi) any combination of two or more thereof.

3. The method of claim 1, wherein the composition reduces (i) the level of one or more acute phase reactants by at least 35%; (ii) the level of absolute neutrophil count (ANC) by at least 25%; (iii) the level of interleukin-1beta (IL-1β) by at least 15%; (iv) the level of NETosis by at least 20%; or (v) any combination of two or more thereof.

4. The method of claim 3, wherein the one or more acute phase reactants comprise C-reactive protein (CRP), ferritin, D-dimer, or a combination of two or more thereof.

5. The method of claim 1, wherein the composition increases (i) the level of total lymphocyte count by at least 20%; (ii) the level of one or more of CD3+T lymphocytes, CD4+T lymphocytes, CD8+T lymphocytes, or combinations thereof by at least 25%; or (iii) both (i) and (ii).

6. The method of claim 1, wherein the composition allows the subject in need thereof to (i) avoid use of a ventilator or (ii) be removed from a ventilator within 72 hours of the administering.

7. The method of claim 1, wherein the composition comprises the bone morphogenetic protein 1 (BMP-1), stem cell growth factor 1 (SCF1), insulin-like growth factor 2 (IGF-2), TIMP-1 (tissue inhibitor of metalloproteinases 1), TIMP-2 (tissue inhibitor of metalloproteinases 2), transforming growth factor β1 (TGF-β1), CD63 antigen, ferritin protein, IGFBP-4 (Insulin-like growth factor binding protein-4), MIF (Macrophage migration inhibitory factor), or macrophage colony stimulating factor (M-CSF), or any combination of two or more thereof.

8. The method of claim 7, wherein the composition comprises the bone morphogenetic protein 1 (BMP-1), stem cell growth factor 1 (SCF1), insulin-like growth factor 2 (IGF-2), TIMP-1 (tissue inhibitor of metalloproteinases 1), TIMP-2 (tissue inhibitor of metalloproteinases 2), transforming growth factor β1 (TGF-β1), CD63 antigen, ferritin protein, IGFBP-4 (Insulin-like growth factor binding protein-4), MIF (Macrophage migration inhibitory factor), and macrophage colony stimulating factor (M-CSF).

9. The method of claim 1, wherein the composition comprises the furin protein, ferritin, PAI-1, or thrombomodulin, or any combination of two or more thereof.

10. The method of claim 1, wherein the composition further comprises hsa-let-7a-5p, hsa-let-7b-5p, hsa-let-7c-5p, hsa-let-7d-3p, hsa-let-7e-5p, hsa-let-7g-5p, hsa-let-7i, hsa-let-7i-5p, hsa-miR-100-5p, hsa-miR-103a-3p, hsa-miR-106a-5p, hsa-miR-106b-5p, hsa-mir-10b, hsa-miR-10b-5p, hsa-mir-1246, hsa-miR-1246, hsa-miR-125a-5p, hsa-miR-125b-5p, hsa-miR-130a-3p, hsa-mir-130b, hsa-miR-130b-3p, hsa-miR-132-3p, hsa-miR-136-5p, hsa-miR-138-5p, hsa-miR-139-5p, hsa-mir-140, hsa-miR-140-3p, hsa-miR-145-5p, hsa-mir-146a, hsa-miR-146a-5p, hsa-miR-148a-3p, hsa-miR-152-3p, hsa-miR-15a-5p, hsa-miR-15b-5p, hsa-mir-16-1, hsa-mir-16-2, hsa-miR-16-5p, hsa-miR-17-5p, hsa-miR-181a-5p, hsa-miR-191-5p, hsa-miR-193a-5p, hsa-miR-193b-3p, hsa-miR-197-3p, hsa-miR-199a-3p, hsa-miR-199a-5p, hsa-miR-199b-5p, hsa-miR-19a-3p, hsa-miR-19b-3p, hsa-miR-20a-5p, hsa-mir-203a, hsa-miR-203a-3p, hsa-miR-214-3p, hsa-mir-21, hsa-miR-21-3p, hsa-miR-21-5p, hsa-mir-221, hsa-miR-221-3p, hsa-mir-222, hsa-miR-222-3p, hsa-miR-22-3p, hsa-miR-23a-3p, hsa-miR-23b-3p, hsa-mir-24-1, hsa-mir-24-2, hsa-miR-24-3p, hsa-mir-25, hsa-miR-25-3p, hsa-miR-26a-5p, hsa-miR-27a-3p, hsa-mir-27b, hsa-miR-27b-3p, hsa-miR-29a-3p, hsa-miR-29c-3p, hsa-miR-30a-5p, hsa-miR-30a-5p, hsa-miR-30b-5p, hsa-miR-30c-5p, hsa-mir-30d, hsa-miR-30d-5p, hsa-mir-30e, hsa-miR-30e-5p, hsa-miR-31-3p, hsa-miR-31-5p, hsa-miR-320a, hsa-miR-342-3p, hsa-miR-345-5p, hsa-miR-34a-5p, hsa-miR-361-5p, hsa-miR-376a-3p, hsa-miR-376c-3p, hsa-miR-423-3p, hsa-miR-423-5p, hsa-miR-424-5p, hsa-miR-484, hsa-mir-486-1, hsa-mir-486-2, hsa-miR-486-5p, hsa-miR-570-3p, hsa-miR-574-3p, hsa-miR-663a, hsa-miR-874-3p, hsa-mir-92a-1, hsa-mir-92a-2, hsa-miR-92a-3p, hsa-miR-92b-3p, hsa-mir-93, hsa-miR-93-5p, hsa-miR-940, hsa-miR-99a-5p, or hsa-miR-99b-5p, or any combination of two or more thereof.

11. The method of claim 10, wherein the composition comprises the hsa-miR-21-5p, miR-24-3p, hsa-miR-222-3p, hsa-miR-27b-3p hsa-miR-125b-5p, has-miR-132-3p, hsa-miR-145-5p, hsa-miR-191-5p, hsa-miR-199a-3p, hsa-miR-221-3p, hsa-miR-22-3p, hsa-miR-23a-3p, hsa-miR-23b-3p, hsa-miR-27a-3p, hsa-miR-29a-3p, hsa-miR-29c-3p, hsa-miR-31-5p, hsa-miR-320a, hsa-miR-34a-5p, hsa-miR-423-3p, hsa-miR-424-5p, or hsa-miR-940, or a combination of two or more thereof.

12. The method of claim 11, wherein the composition comprises the hsa-miR-21-5p, miR-24-3p, hsa-miR-222-3p, and hsa-miR-27b-3p.

13. The method of claim 1, wherein the composition further comprises an oligosaccharide.

14. The method of claim 1, wherein the composition comprises extracellular vesicles with a size of about 20 nm to about 200 nm.

15. The method of claim 1, wherein the composition comprises from about 0.00001 wt % to about 20 wt % of extracellular vesicles.

16. The method of claim 1, wherein the extracellular vesicles are present in the composition (i) in an amount between about 1 million and about 100 billion vesicles per mL, when formulated as a liquid composition or (ii) in an amount between about 10 million and about 1 trillion, when formulated as a lyophilized powder composition.

17. The method of claim 1, wherein the administering is via parenteral administration.

18. The method of claim 1, wherein the composition is prepared in a method comprising culturing bone marrow mesenchymal stem cells (MSCs) at an oxygen level of 0.1% to 5%.

19. The method of claim 18, wherein the culturing occurs at a pH of 6.0 to 6.9.
